(19) United States
(12) Patent Application Publication
(10) Pub. No.: US 2019/0380329 A1
(43) Pub. Date: Dec. 19, 2019

(54) PERFUSION DEVICE AND PERFUSION METHOD

(71) Applicant: SYSMEX CORPORATION, Kobe-shi (JP)

(72) Inventors: Nobuyasu HORI, Kobe-shi (JP); Shingo FUJIYAMA, Kobe-shi (JP)

(73) Assignee: SYSMEX CORPORATION, Kobe-shi (JP)

(21) Appl. No.: 16/552,519

(22) Filed: Aug. 27, 2019

Related U.S. Application Data
(63) Continuation of application No. PCT/JP2018/007449, filed on Feb. 28, 2018.

(30) Foreign Application Priority Data
Feb. 28, 2017 (JP) 2017-036263

(51) Int. Cl. A01N 1/02; C12N 5/078

(52) U.S. Cl. CPC A01N 1/0247; C12N 2506/11; C12N 5/0644; A01N 1/0226

(57) ABSTRACT

The present invention relates to a perfusion device and a perfusion method for culturing an undifferentiated cell using an organ or tissue extracted from a living body.

### CROSS REFERENCE TO RELATED APPLICATIONS

This application is a continuation application of International Application No. PCT/JP2018/007449, filed on Feb. 28, 2018, which claims priority to Japanese Patent Application No. 2017-036263, filed on Feb. 28, 2017. The disclosures of these applications are hereby incorporated by reference.

### BACKGROUND OF THE INVENTION

### Field of the Invention

The present invention relates to a perfusion device and a perfusion method.

### Description of the Background Art

A technology for differentiating undifferentiated cells such as stem cells into desired cells has been expected as a technology applicable to regenerative medicine, and the like. As a method for differentiating a cell, an in vitro culture system is usually used by culturing undifferentiated cells in a culture vessel and adding a factor that induces differentiation. However, it takes several days to obtain desired differentiated cells. In recent years, the present inventors have succeeded in differentiating cells in a shorter period by an ex vivo culture system using an organ extracted from a living body. For example, US 2015/0275176 A describes a method using perfusion of an organ extracted from a living body. In this method, undifferentiated cells are introduced into a perfused organ, and the cells are differentiated inside the organ and recovered.

### SUMMARY OF THE INVENTION

However, considering an industrial use of differentiated cells, it is desirable to further improve the yield of differentiated cells in an ex vivo culture system using an organ or tissue.

Therefore, the present invention provides a perfusion device including a storage unit in which an organ or tissue extracted from a living body is stored; a fluid feeding unit configured to feed a fluid containing an undifferentiated cell into the organ or tissue stored in the storage unit; a recovery unit configured to recover a fluid containing a cell differentiated from the undifferentiated cell; a first conduit connecting the organ or tissue stored in the storage unit and the fluid feeding unit; a second conduit connecting the organ or tissue stored in the storage unit and the recovery unit; and a pressure regulating unit provided in the second conduit. In this perfusion device, the pressure regulating unit regulates pressure inside the organ or tissue to be positive when the fluid feeding unit feeds the fluid containing the undifferentiated cell into the organ or tissue.

The present invention also provides a perfusion method including the steps of feeding, under positive pressure, a fluid containing an undifferentiated cell into an organ or tissue extracted from a living body; culturing the undifferentiated cell inside the organ or tissue; and recovering a fluid containing a cell differentiated from the undifferentiated cell from the organ or tissue.

Furthermore, the present invention provides a perfusion method including the steps of feeding a fluid containing an undifferentiated cell into an interior of a processed bone having an outer surface of a bone covered with a covering agent that adheres to the outer surface of the bone, and a hole that penetrates through the covering agent and the outer surface of the bone and reaches an interior of the bone; culturing the undifferentiated cell in the interior of the processed bone; and recovering a fluid containing a platelet differentiated from the undifferentiated cell from the interior of the processed bone.

The present invention provides a perfusion device including a storage unit in which an organ or tissue extracted from a living body is stored; a first fluid feeding unit configured to feed a fluid containing an undifferentiated cell into an organ or tissue stored in the storage unit; a recovery unit configured to recover a fluid containing a cell differentiated from the undifferentiated cell; a first conduit connecting the organ or tissue stored in the storage unit and the first fluid feeding unit; a second conduit connecting the organ or tissue stored in the storage unit and the recovery unit; and a second fluid feeding unit provided in the second conduit. In this perfusion device, the second fluid feeding unit feeds the fluid containing the undifferentiated cell derived out from the organ or tissue into the organ or tissue by reverse fluid feed.

### BRIEF DESCRIPTION OF THE DRAWINGS

FIG. 1 is a schematic view showing a configuration of a perfusion device.

FIG. 2 is a flow chart showing a procedure for introducing a cell and recovering a cell.

FIG. 3 is a circuit diagram showing an operation state of a perfusion system of a perfusion device when an organ is washed.

FIG. 4 is a circuit diagram showing an operation state of a perfusion system of a perfusion device when an organ is perfused.

FIG. 5 is a circuit diagram showing an operation state of a perfusion system of a perfusion device when an undifferentiated cell is introduced into an organ.

FIG. 6 is a circuit diagram showing an operation state of a perfusion system of a perfusion device when cell culture inside an organ is performed.

FIG. 7 is a circuit diagram showing an operation state of a perfusion system of a perfusion device when a cell is recovered from an organ.

FIG. 8 is a circuit diagram showing an operation state of a perfusion system of a perfusion device when the device is washed.

FIG. 9 is a schematic view showing a configuration of a perfusion device used in Experimental Example 1.

FIG. 10A is a graph showing a ratio of CD42b-expressing cells in platelet-sized cells derived from megakaryocytic cells introduced into a processed bone or a tube.

FIG. 10B is a graph showing a ratio of CD61-expressing cells in platelet-sized cells derived from megakaryocytic cells introduced into a processed bone or a tube.

FIG. 11 is a schematic view showing a configuration of a perfusion device used in Experimental Example 3.

FIG. 12A is a graph showing a ratio of CD42b-expressing cells in platelet-sized cells derived from megakaryocytic cells introduced into spleen or a tube.

FIG. 12B is a graph showing a ratio of CD61-expressing cells in platelet-sized cells derived from megakaryocytic cells introduced into spleen or a tube.

FIG. 13 is a graph showing results of extracting a population of CD42b-positive platelets by flow cytometry (FCM) analysis.

FIG. 14 is a graph showing results of extracting a population of CD61-positive platelets by FCM analysis.

FIG. 15 is a graph showing results of extracting a population of PAC-1-positive platelets by FCM analysis.

FIG. 16A is a graph showing fluctuations in pressure inside the processed bone between start and end of introduction of perfusate in the state where a lead-out hole is closed.

FIG. 16B is a graph showing fluctuations in pressure inside the processed bone between start and end of introduction of perfusate in the state where a lead-out hole is closed.

FIG. 17 is a schematic view showing a configuration of a perfusion device equipped with two fluid feeding pumps.

FIG. 18 is an immunostained image showing megakaryocytes and platelets derived from megakaryocytic cells cultured in a femur.

### DESCRIPTION OF THE PREFERRED EMBODIMENTS

### [1. Perfusion Device]

### (Configuration of Perfusion Device)

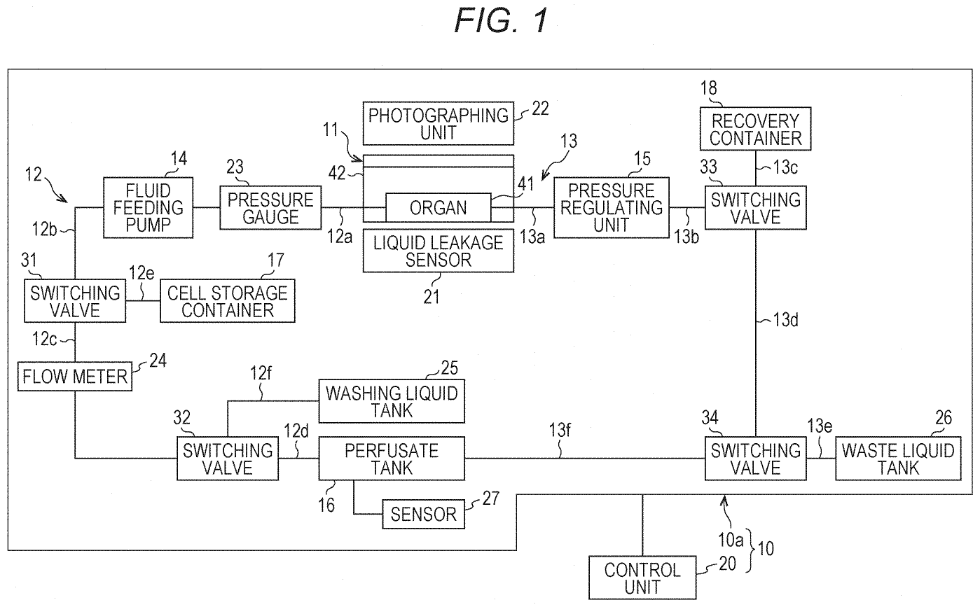

As used herein, perfusion refers to introducing a liquid into an organ or tissue extracted from a living body and deriving the liquid from the organ or tissue. An example of a perfusion device according to the present embodiment will be described below with reference to the drawings. However, the present embodiment is not limited to this example. The perfusion device of the present embodiment can be used for the perfusion method described later. As shown in FIG. 1, a perfusion device 10 includes a perfusion system 10a and a control unit 20. When a user directly operates a fluid feeding pump 14, a pressure regulating unit 15, and switching valves 31, 32, 33 and 34 described later, the perfusion device 10 may not include the control unit 20.

The perfusion system 10a includes a container 11 for disposing an organ 41, a first conduit 12 for introducing liquid into the organ 41, a second conduit 13 for deriving the liquid from the organ 41, the fluid feeding pump 14, the pressure regulating unit 15, a perfusate tank 16 for storing a perfusate, a cell storage container 17 for storing undifferentiated cells, and a recovery container 18 for recovering a fluid containing a cell differentiated from the undifferentiated cell. In the present embodiment, the perfusion system 10a further includes a liquid leakage sensor 21, an imaging unit 22, a pressure gauge 23, a flow meter 24, a washing liquid tank 25 for storing the washing solution of the device, a waste liquid tank 26 for recovering a waste liquid, and a sensor 27 connected to the perfusate tank 16.

The container 11 constitutes a storage unit of the perfusion device of the present embodiment. The container 11 should be a container which can store the organ 41 extracted from a living body and a preservation solution 42. In the following, instead of the organ 41, a tissue extracted from a living body may be disposed in the container 11. The container 11 may be an open type container having an opening or may be a sealable container. The capacity of the container 11 should have a capacity sufficient to store the organ 41 and the preservation solution 42. It is preferable that the organ 41 and the preservation solution 42 stored in the container 11 are maintained at a predetermined temperature. Therefore, the container 11 may be disposed in an incubator that can maintain a predetermined temperature. Alternatively, an outer periphery of the container 11 may be covered with a heat retention unit that can maintain a predetermined temperature. Moreover, it is preferable that the organ 41 and the preservation solution 42 stored in the container 11 are maintained at a predetermined carbon dioxide concentration. Thus, the container 11 may be disposed in an incubator that can maintain a predetermined carbon dioxide concentration.

The liquid leakage sensor 21 may be installed in the container 11. The liquid leakage sensor 21 monitors the presence or absence of leakage of the preservation solution from the container 11. The liquid leakage sensor 21 may be provided on the outer periphery of the container 11, or may be provided separately from the container 11 within a range where the presence or absence of leakage of the preservation solution can be monitored.

The photographing unit 22 may be installed in the container 11. The photographing unit 22 monitors a change in the state of the organ 41. The photographing unit 22 may be installed at a position where the organ 41 in the container 11 can be monitored.

The first conduit 12 is a pipe serving as a flow path of the liquid introduced into the organ 41. The first conduit 12 includes conduits 12a, 12b, 12c, 12d, 12e and 12f. The conduit 12a connects the organ 41 and the fluid feeding pump 14. The conduit 12b connects the fluid feeding pump 14 and the switching valve 31. The conduit 12c connects the switching valves 31 and 32. The conduit 12d connects the switching valve 32 and the perfusate tank 16. The conduit 12e connects the switching valve 31 and the cell storage container 17. The conduit 12f connects the switching valve 32 and the washing liquid tank 25.

When the organ 41 is disposed in the container 11, the first conduit 12 connects the organ 41 in the container 11 to the fluid feeding pump 14 and the perfusate tank 16. In this case, the first conduit 12 functions as a flow path for introducing the perfusate into the organ 41. Further, when a fluid containing an undifferentiated cell is introduced into the organ 41, the first conduit 12 connects the organ 41 in the container 11 to the fluid feeding pump 14 and the cell storage container 17. In this case, the first conduit 12 functions as a flow path for introducing the fluid containing the undifferentiated cell into the organ 41. When the organ 41 is not disposed in the container 11, the first conduit 12 connects the container 11 to the fluid feeding pump 14 and the washing liquid tank 25. In this case, the first conduit 12 functions as a flow path for introducing the washing liquid in the device into the container 11.

The first conduit 12 includes the pressure gauge 23 and the flow meter 24. The pressure gauge 23 measures pressure inside the first conduit 12. When the second conduit 13 is closed by the pressure regulating unit 15 described later, the pressure measured by the pressure gauge 23 reflects pressure inside the organ 41. The flow meter 24 measures flow rate and/or flow velocity in the first conduit 12.

The first conduit 12 includes the switching valves 31 and 32. The switching valve 31 switches a fluid to be introduced into the container 11 (a fluid containing an undifferentiated cell in the cell storage container 17 or a perfusate in the perfusate tank 16). The switching valve 32 switches a liquid to be introduced into the container 11 (the perfusate in the perfusate tank 16 or a washing liquid in the washing liquid tank 25). The switching valve includes a multi-way stopcock such as a three-way stopcock, a solenoid valve, and the like, but it is not particularly limited.

The perfusate tank 16 stores a perfusate for introduction into the organ 41. The sensor 27 is provided in the perfusate tank 16. The sensor 27 measures the state of the perfusate in the perfusate tank 16, for example, pH, temperature, dissolved oxygen amount, redox potential, and the like. The washing liquid tank 25 stores a washing liquid for washing the conduits 12a, 12b, 12c, 12d, 12e, 13a, 13b, 13c, 13d, 13e and 13f, the sensor 27, the switching valves 31, 32, 33 and 34, and the like in the device.

The cell storage container 17 stores a fluid containing an undifferentiated cell for introduction into the organ 41. The cell storage container 17 should be any container that can hold the undifferentiated cell in a viable state. The cell storage container 17 may be maintained at a predetermined temperature and carbon dioxide concentration suitable for survival of the undifferentiated cell. The cell storage container 17 may be disposed in an incubator that can maintain a predetermined temperature and carbon dioxide concentration.

The fluid feeding pump 14 feeds the perfusate in the perfusate tank 16, the fluid containing the undifferentiated cell in the cell storage container 17, or the device washing liquid in the washing liquid tank 25. The fluid feeding pump includes a tubing pump, an electromagnetic pump, and the like, but it is not particularly limited. In the present embodiment, the fluid feeding pump 14, the perfusate tank 16 and the cell storage container 17 constitute a fluid feeding unit of the perfusion device.

The second conduit 13 is a pipe serving as a flow path of the liquid derived out from the organ 41. The second conduit 13 includes conduits 13a, 13b, 13c, 13d, 13e and 13f. The conduit 13a connects the organ 41 and the pressure regulating unit 15. The conduit 13b connects the pressure regulating unit 15 and the switching valve 33. The conduit 13c connects the switching valve 33 and the recovery container 18. The conduit 13d connects the switching valves 33 and 34. The conduit 13e connects the switching valve 34 and the waste liquid tank 26. The conduit 13f connects the switching valve 34 and the perfusate tank 16.

When the organ 41 is disposed in the container 11, the second conduit 13 connects the organ 41 in the container 11 to the perfusate tank 16 or the waste liquid tank 26. In this case, the second conduit 13 functions as a flow path for the perfusate derived out from the organ 41. Further, when a fluid containing an undifferentiated cell is introduced into the organ 41, the second conduit 13 connects the organ 41 in the container 11 and the recovery container 18. In this case, the second conduit 13 functions as a flow path for a fluid containing a cell differentiated from the undifferentiated cell derived out from the organ 41. When the organ 41 is not disposed in the container 11, the second conduit 13 connects the container 11 and the waste liquid tank 26. In this case, the second conduit 13 functions as a flow path for the washing liquid derived out from the container 11.

The second conduit 13 includes a pressure regulating unit 15. The pressure regulating unit 15 connects to the organ 41 by way of the conduit 13a. The pressure regulating unit 15 regulates so that the pressure inside the organ 41 is positive when the fluid feeding pump 14 introduces a fluid containing an undifferentiated cell into the organ 41. Here, the phrase “the pressure inside the organ or tissue is positive” refers that the flow rate on the recovery side is lower than the flow rate on the fluid feeding side, based on the same flow rate on the fluid feeding side and the flow rate on the recovery side. For example, the pressure regulating unit 15 regulates pressure applied into the organ 41 by changing the flow rate (or flow velocity) in the second conduit 13. When introducing the perfusate or the fluid containing the undifferentiated cell into the organ 41, the pressure regulating unit 15 makes the flow rate (or flow velocity) in the second conduit 13 lower than the flow rate (or flow velocity) in the first conduit 12 to apply positive pressure into the organ 41. When the fluid feeding pump 14 introduces the fluid containing the undifferentiated cell into the organ 41, the pressure regulating unit 15 may close the second conduit 13 to adjust so that liquid is not derived out from the organ 41. Thus, the pressure regulating unit 15 can regulates pressure so as to provide a time for setting the pressure inside the organ 41 to a positive pressure (for example, a pressure of 5 kPa or more and 100 kPa or less, and preferably 10 kPa or more and 75 kPa or less) when the fluid feeding pump 14 introduces the fluid containing the undifferentiated cell into the organ 41. The pressure regulating unit 15 restores the pressure inside the organ 41 by restoring the flow rate (or flow velocity) in the second conduit 13. The pressure regulating unit 15 is not particularly limited as long as the flow rate (or flow velocity) in the second conduit 13 can be adjusted, and examples thereof include valves such as a three-way stopcock, a two-way stopcock, and a solenoid valve.

The second conduit 13 includes the switching valves 33 and 34. The switching valve 33 switches the flow path (conduit 13c or 13d) of the fluid derived out from the organ 41. The switching valve 34 switches the flow path (conduit 13e or 130 of the fluid flowing through the conduit 13d.

The recovery container 18 is a container for storing the fluid containing the cell differentiated from the undifferentiated cell derived out from the organ 41. The recovery container 18 should be any container that can hold the differentiated cell in a viable state. The recovery container 18 may be maintained at a predetermined temperature and carbon dioxide concentration suitable for survival of the differentiated cell. The recovery container 18 may be disposed in an incubator that can maintain a predetermined temperature and carbon dioxide concentration. In the present embodiment, the recovery container 18 constitutes a recovery unit of the perfusion device.

The control unit 20 is connected to the perfusion system 10a. The control unit 20 can control an operation of the fluid feeding pump 14 and the pressure regulating unit 15, based on the information on the presence or absence of solution leakage obtained in the liquid leakage sensor 21, the information on the state of the organ obtained in the photographing unit 22, the information on the pressure inside the flow path obtained in the pressure gauge 23, the information on the flow rate obtained in the flow meter 24, and the like. Further, the control unit 20 can control the switching valves 31, 32, 33 and 34, in accordance with the procedure of the perfusion method described later.

### (Operation of Perfusion Device)

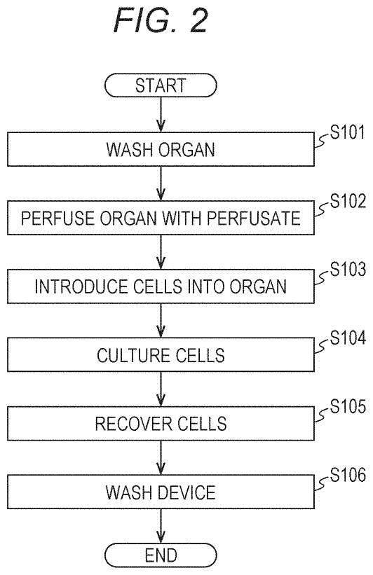

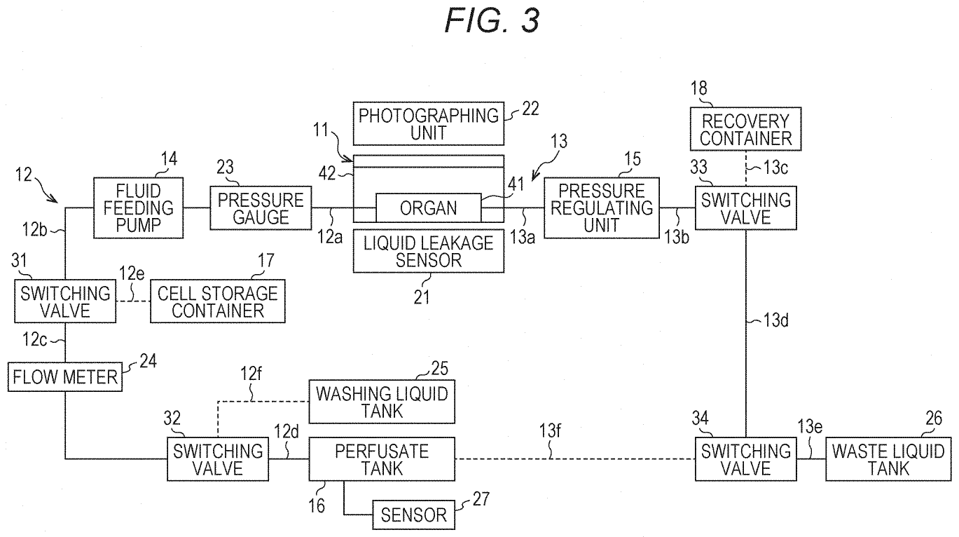

An example of the operation of the perfusion device of the present embodiment will be described with reference to the drawings. However, the present embodiment is not limited to this example. With reference to FIG. 2, the procedure in the case of performing cell introduction to an organ, and cell recovery from an organ with the perfusion device of the present embodiment will be described. The flow paths of liquid in each step of FIG. 2 are shown in FIGS. 3 to 8. In these figures, a conduit indicated by a solid line is a flow path of the liquid, and no liquid flows in a conduit indicated by a broken line.

With reference to FIG. 2, in step S101, the organ is washed with the perfusate. As shown in FIG. 3, the conduit 12d connected to the perfusate tank 16 is connected to the conduit 12c by way of the switching valve 32. Further, the conduit 12c is connected to the conduit 12b by way of the switching valve 31. The conduit 12b is connected to the conduit 12a by way of the fluid feeding pump 14. The conduit 12a is connected to an artery of the organ 41 stored in the container 11. The conduit 13a is connected to a vein of the organ 41 stored in the container 11. The perfusate in the perfusate tank 16 is introduced into the organ 41 through the first conduit 12, by the fluid feeding pump 14. Then, the perfusate is derived out from the organ through the conduit 13a and recovered into the waste liquid tank 26 through the conduits 13b, 13d and 13e. Thus, blood cells and the like inside the organ can be removed prior to introduction of the undifferentiated cell.

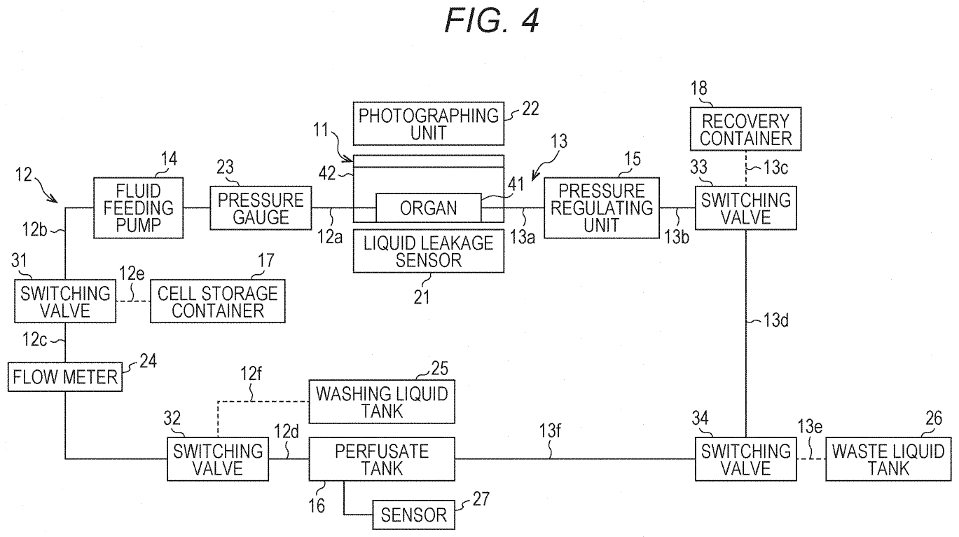

In step S102, the organ is perfused with the perfusate. The flow path of the liquid in step S102 is shown in FIG. 4. In FIG. 4, as in FIG. 3, the perfusate tank 16 and the fluid feeding pump 14 are connected by way of the conduits 12b, 12c and 12d and the switching valve 31. The fluid feeding pump 14 and the organ 41 are connected by way of the conduit 12a. Also, the conduit 13a is connected to the vein of the organ 41 stored in the container 11. In step S102, as in step S101, the perfusate in the perfusate tank 16 is introduced into the organ 41 through the first conduit 12 by the fluid feeding pump 14. Then, the perfusate is derived out from the organ through the conduit 13a and returned to the perfusate tank 16 through the conduits 13b, 13d and 13f. In step S102, the pressure inside the flow path, the pressure applied to the organ, and the flow rate (or flow velocity) of the perfusate may be monitored by the pressure gauge 23 and the flow meter 24.

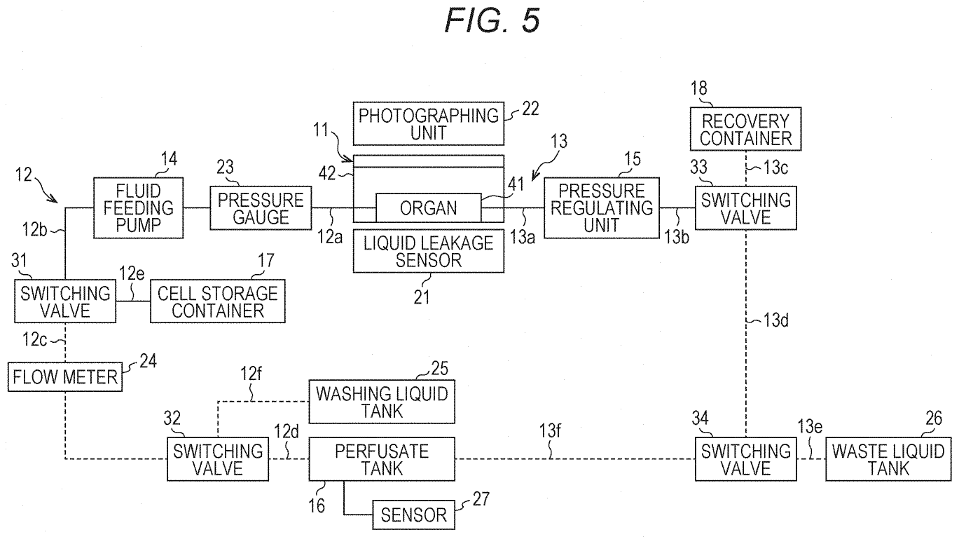

In step S103, undifferentiated cells are introduced into the organ. The flow path of the liquid in step S103 is shown in FIG. 5. As shown in FIG. 5, the conduit 12e connected to the cell storage container 17 storing the fluid containing the undifferentiated cell is connected to the conduit 12b by way of the switching valve 31. At this time, an opening on the conduit 12c side in the switching valve 31 is closed. The conduit 12b is connected to the conduit 12a by way of the fluid feeding pump 14. The conduit 12a is connected to the artery of the organ 41 stored in the container 11. The conduit 13a is connected to the vein of the organ 41 stored in the container 11. Openings on the conduit 13c side and the conduit 13d side in the switching valve 33 are closed. The fluid containing the undifferentiated cell in the cell storage container 17 is introduced into the organ 41 through the first conduit 12, by the fluid feeding pump 14. At this time, the pressure regulating unit 15 can apply positive pressure into the organ 41 by introducing the fluid containing the undifferentiated cell, in a state where the flow rate (or flow velocity) in the conduit 13a is reduced. The pressure regulating unit 15 may close the conduit 13a. By making the pressure inside the organ 41 positive, the undifferentiated cell can be diffused and permeated throughout the organ 41. In step S103, the pressure inside the flow path, the pressure applied to the organ, and the flow rate (or flow velocity) of the perfusate may be monitored by the pressure gauge 23 and the flow meter 24.

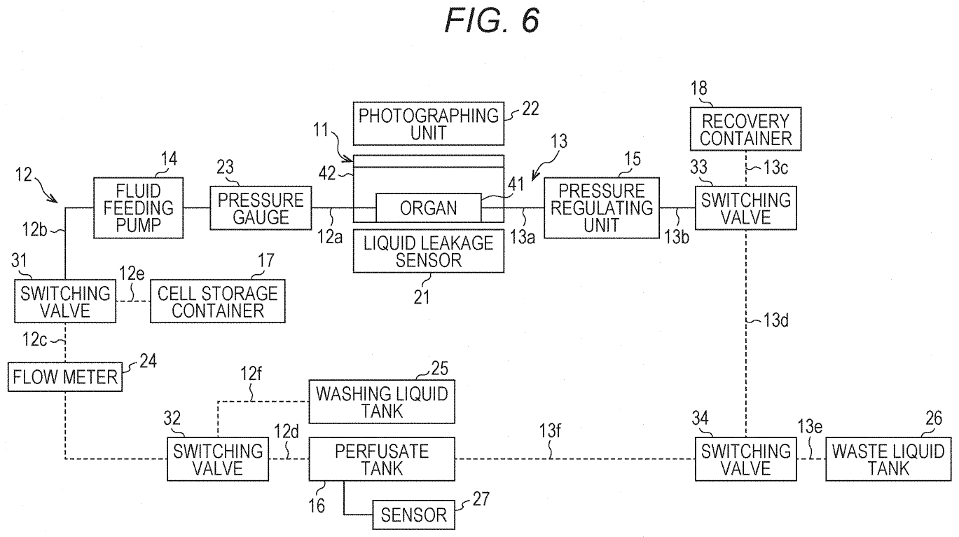

In step S104, cell culture inside the organ is performed. The flow path of the liquid in step S104 is shown in FIG. 6. Openings on the conduit 12c side and the conduit 12e side in the switching valve 31 are closed. Moreover, openings on the conduit 13c side and the conduit 13d side in the switching valve 33 are closed. At this time, the fluid feeding pump 14 is not operating. Thus, the undifferentiated cell is cultured inside the organ 41.

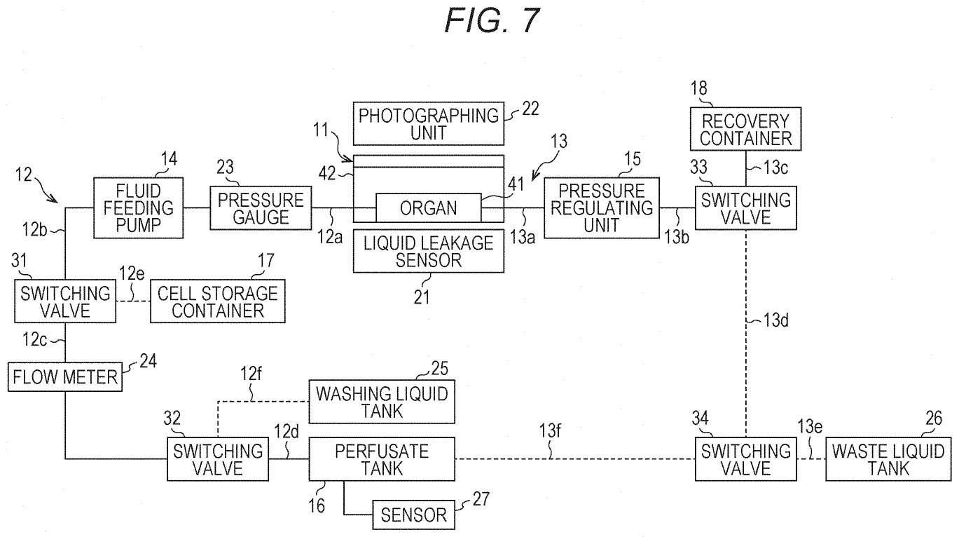

In step S105, the cell is recovered. Here, the terms “recovery” and “recover” include not only transferring the differentiated cell or a fluid containing the cell from the organ or tissue into the recovery container, but also simply taking out the differentiated cell or the fluid containing the cell from the interior of the organ or tissue to the outside. The flow path of the liquid in step S105 is shown in FIG. 7. In FIG. 7, as in FIG. 3, the perfusate tank 16 and the fluid feeding pump 14 are connected by way of the conduits 12b, 12c and 12d and the switching valve 31. The fluid feeding pump 14 and the organ 41 are connected by way of the conduit 12a. Also, the conduit 13a is connected to the vein of the organ 41 stored in the container 11. In step S105, as in step S101, the perfusate in the perfusate tank 16 is introduced into the organ 41 through the first conduit 12 by the fluid feeding pump 14. Then, a fluid containing the cultured cell is derived out from the organ 41 through the conduit 13a, and is recovered into the recovery container 18 through the conduits 13b and 13c. At this time, the recovered fluid contains the cell differentiated from undifferentiated cell. In step S105, the pressure inside the flow path, the pressure applied to the organ, and the flow rate (or flow velocity) of the perfusate may be monitored by the pressure gauge 23 and the flow meter 24.

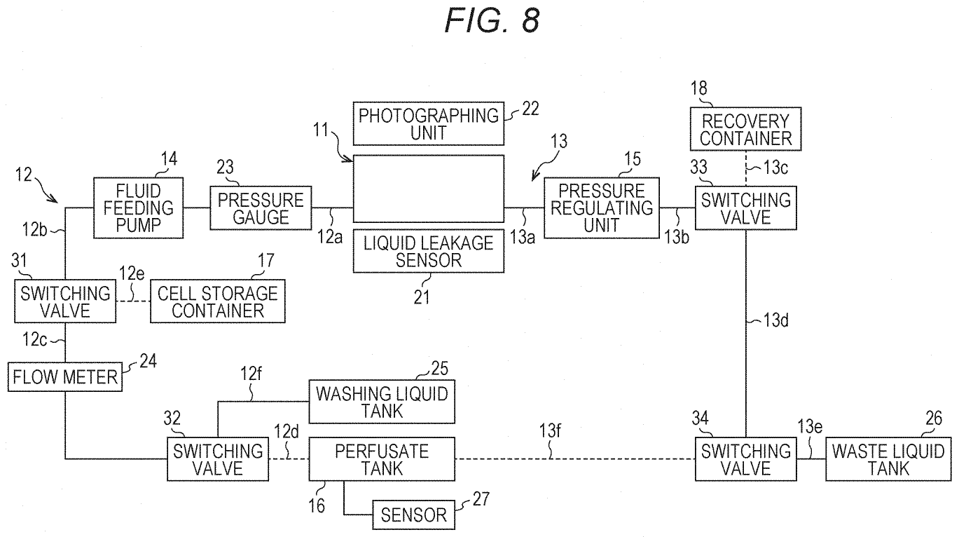

In step S106, the perfusion device is washed. At this time, the organ 41 is extracted from the container 11. The flow path of the liquid in step S106 is shown in FIG. 8. As shown in FIG. 8, the conduit 12f connected to the washing liquid tank 25 is connected to the conduit 12c by way of the switching valve 32. Further, the conduit 12c is connected to the conduit 12b by way of the switching valve 31. The conduit 12b is connected to the conduit 12a by way of the fluid feeding pump 14. The conduit 12a is connected to the container 11. The conduit 13a is connected to the container 11. The washing liquid in the washing liquid tank 25 is introduced into the container 11 through the first conduit 12, by the fluid feeding pump 14. Then, the washing liquid is derived out from the container 11 through the second conduit 13 and recovered into the waste liquid tank 26 through the conduits 13b, 13d and 13e.

In a further embodiment, the fluid feeding pump 14 may be able to feed fluid in the reverse direction (hereinafter also referred to as “reverse fluid feed”). In the case of normal fluid feed, the fluid feeding pump 14 feeds liquid so that liquid travels from the first conduit through the organ or tissue into the second conduit. In the case of reverse fluid feed, the fluid feeding pump 14 feeds liquid so that liquid travels from the second conduit through the organ or tissue into the first conduit. Once a fluid containing the undifferentiated cell is introduced into the organ or tissue and then reversely fed, the introduced fluid containing the undifferentiated cell reciprocates within the organ or tissue. Thus, the undifferentiated cell can be further diffused into the organ or tissue. The feed and reverse feed of the fluid containing the undifferentiated cell may be repeated. Examples of the fluid feeding pump capable of performing reverse fluid feed include a tubing pump capable of switching the fluid feeding direction.

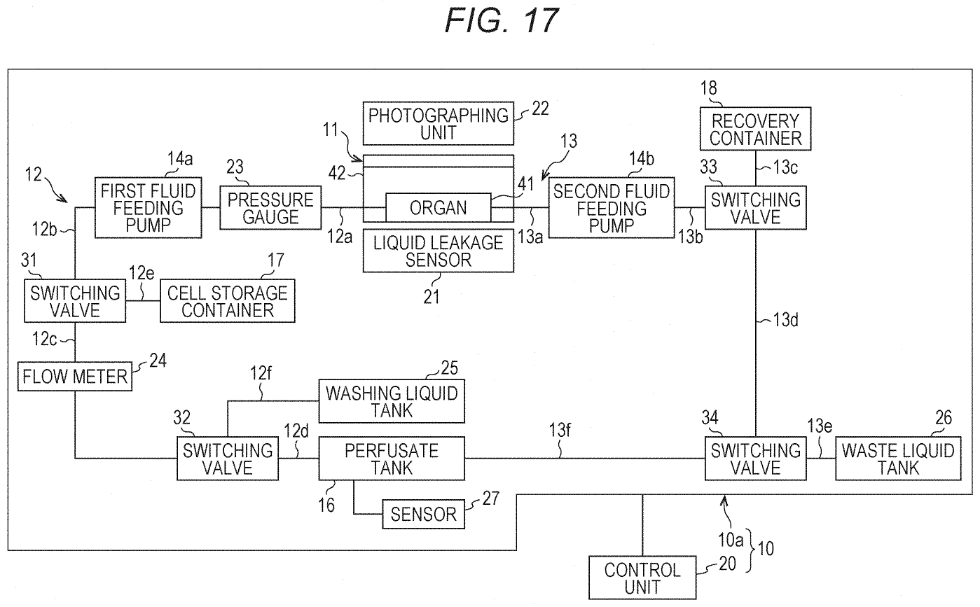

In another embodiment, the perfusion device may include two fluid feeding pumps. An example of the perfusion device according to this embodiment will be described with reference to FIG. 17. However, the present embodiment is not limited only to this example. The perfusion device 10 shown in FIG. 17 is the same as the perfusion device 10 shown in FIG. 1 except that the second conduit 13 includes the second fluid feeding pump 14b and does not include the pressure regulating unit 15. The second fluid feeding pump 14b may be installed at any position of the second conduit 13, but is preferably connected between the organ 41 and the switching valve 33.

The first fluid feeding pump 14a is the same as the fluid feeding pump 14 shown in FIG. 1, and feeds the perfusate in the perfusate tank 16, the fluid containing the undifferentiated cell in the cell storage container 17, or the device washing liquid in the washing liquid tank 25. The second fluid feeding pump 14b feeds liquid in the same manner as the first fluid feeding pump 14a in the steps of washing the organ or tissue, perfusing the perfusate, recovering the cell, and washing the perfusion device. In the step of introducing the undifferentiated cell into the organ or tissue, the second fluid feeding pump 14b introduces a fluid containing an undifferentiated cell derived out from the interior of the organ or tissue into the organ 41 again by reverse fluid feed. Therefore, the second fluid feeding pump 14b is preferably a pump capable of switching the fluid feeding direction.

In the step of introducing the undifferentiated cell into the organ or tissue, the fluid containing the undifferentiated cell reciprocates within the organ or tissue by alternately performing fluid feed by the first fluid feeding pump 14a and reverse fluid feed by the second fluid feeding pump 14b. Thus, the undifferentiated cell can be further diffused into the organ or tissue.

The operation of the perfusion device shown in FIG. 17 in the step of introducing the undifferentiated cell is, for example, as follows. The fluid containing the undifferentiated cell in the cell storage container 17 is introduced into the organ 41 through the first conduit 12, by the first fluid feeding pump 14a. When the cell is introduced into the organ 41 by the first fluid feeding pump 14a, the second fluid feeding pump 14b may be stopped. Alternatively, the second fluid feeding pump 14b may feed liquid in the same direction as the first fluid feeding pump 14a. By further continuing the fluid feed, the fluid containing the undifferentiated cell is derived out from the organ 41 to the second fluid feeding pump 14b through the conduits 13a and 13b. Then, the first fluid feeding pump 14a is stopped, and the second fluid feeding pump 14b is operated to perform reverse fluid feed. Thus, the fluid containing the undifferentiated cell derived out from the organ 41 is introduced into the organ 41 again. After reintroduction of the fluid containing the undifferentiated cell by reverse fluid feed, fluid feed by the first fluid feeding pump 14a and reverse fluid feed by the second fluid feeding pump 14b may be repeated. Alternatively, the cell may be cultured.

### [2. Perfusion Method]

In the perfusion method of the present embodiment, a fluid containing an undifferentiated cell is introduced into an organ or tissue extracted from a living body, and the cell is cultured inside the organ to recover the fluid containing the differentiated cell. Therefore, the perfusion method of the present embodiment can also be interpreted as a method of obtaining a cell differentiated from an undifferentiated cell.

The organ used for the perfusion device of the present embodiment is not particularly limited as long as it is an organ extracted from a living body. The organ may be a solid organ or a hollow organ. Examples of the solid organ include spleen, heart, liver, lung, pancreas, kidney, brain, and the like. Examples of the hollow organ include small intestine, large intestine, rectum, uterus, bladder, and the like. Among them, solid organs are preferred, and spleen, heart, liver, lung, pancreas and kidney are particularly preferred. A supply source of the organ is not particularly limited, but an organ extracted from an animal excluding human is preferable. Examples of such animals include pigs, cows, horses, goats, sheep, monkeys, dogs, cats, rabbits, guinea pigs, rats, mice, chickens, and the like.

The tissue used for the perfusion device of the present embodiment is not particularly limited as long as it is a tissue extracted from a living body, and examples include bone, cartilage, muscle, blood vessel, trachea, and the like. A supply source of the tissue is not particularly limited, and, for example, a tissue extracted from an animal excluding human is preferable. In the present embodiment, it is preferable to use bone as the tissue.

The type of bone is not particularly limited, and any type of bone such as long bones, short bones, flat bones and irregular bones may be used. Examples of the long bones include humerus, radius, ulna, metacarpal, femur, tibia, fibula, metatarsus, and the like. Examples of the short bones include carpus, tarsus, and the like. Examples of the flat bones include parietal bone, sternum, rib, ilium, pubis, ischium, and the like. Examples of the irregular bones include vertebra, scapula, and the like. Among them, femur, humerus, sternum, pubis, ilium, rib and vertebra are preferable.

In the present embodiment, the organ or tissue extracted from a living body may be used as it is, or the organ or tissue may be treated or processed so as to be suitable for perfusion. For example, when perfusion is performed by introducing liquid from an artery of an organ and deriving the liquid from a vein, the artery into which the liquid is introduced and blood vessels other than the vein from which the liquid is derived out may be ligated. When using an organ or tissue which does not have a site into which liquid can be introduced and derived out, a hole or the like may be formed for introducing and deriving liquid on the organ or tissue.

In the present embodiment, it is particularly preferable to use a bone processed to be suitable for perfusion (hereinafter, also referred to as “processed bone”). A processed bone has an outer surface of the bone covered with a covering agent attached closely to the outer surface of the bone, and a hole that penetrates through the covering agent and the outer surface of the bone and reaches the interior of the bone. Such a processed bone can be prepared with reference to JP 2015-228848 A. Specifically, a processed bone can be prepared by coating a bone with a covering agent and forming a hole in the bone. The order of coating a bone with a covering agent and forming a hole is not limited, and either one may be performed first.

The hole that penetrates through the covering agent and the outer surface of the bone and reaches the interior of the bone is a hole for introducing liquid into the bone and for deriving liquid from the bone. The number of the holes may be one or more. When there are two holes, for example, one may be a hole for introducing liquid (hereinafter referred to as an introduction hole), and the other may be a hole for deriving liquid (hereinafter referred to as a lead-out hole). The number of the introduction holes and the lead-out holes may be the same as or different from each other. When there is one hole in the processed bone, the hole serves as both an introduction hole and a lead-out hole.

The position of the hole in the bone is not particularly limited. For example, when opening a hole in a portion covered with periosteum, such as diaphysis of a long bone, the hole is a hole that penetrates through the covering agent and the outer surface of the bone and reaches the interior of the bone. There is no periosteum on an articular surface of bone, and the articular surface is covered with articular cartilage. Therefore, when opening a hole in the articular surface of bone, the hole is a hole that penetrates through the covering agent and the outer surface of the bone and reaches the interior of the bone.

The depth of the hole is preferably a depth such that liquid introduced from the hole can be brought into contact with bone marrow. Such depth of the hole is, for example, a depth reaching ossein, preferably a depth reaching spongin cancellous bone, and more preferably a depth reaching medullary cavity. The size of the hole should be a size that allows introduction and lead-out of a fluid containing a cell. For example, the diameter of the hole is the same diameter as a pipe or injection needle used for introduction and lead-out of a fluid containing a cell.

Preferably, there is a fixed distance between the introduction hole and the lead-out hole depending on the size of the bone. For example, in the case of a long bone such as a femur, an introduction hole may be formed in a diaphyseal portion close to one epiphysis, and a lead-out hole may be formed in a diaphyseal portion close to the opposite epiphysis.

The covering agent is attached closely to the outer surface of the bone in order to suppress the leakage of the liquid and cells, which are to be recovered from the lead-out hole, from the outer surface of the bone. Examples of such covering agent include resins, adhesives, polymeric membranes, gels, gypsum, and the like known in the art. The covering agent may be used alone or in combination of two or more. In the present specification, also in the case where the outer surface of a bone to which a piece of meat or the like is attached closely is covered with a covering agent, it is included in that the covering agent “attached closely to the outer surface of the bone”.

A curable resin, a plastic resin or the like can be used for a resin as a covering agent. Examples of the curable resin include thermosetting resins, photocurable resins, and the like. Examples of the plastic resin include thermoplastic resins, and the like. Examples of the thermosetting resin include epoxy resins, silicone resins, phenol resins, urea resins, melamine resins, unsaturated polyester resins, phenoxy resins, vinyl ester resins, furan resins, diallyl phthalate resins, and the like.

Examples of the thermoplastic resins include polyvinyl chloride, polyvinylidene chloride, polystyrene, styrene-acrylonitrile copolymer, high density polyethylene, medium density polyethylene, low density polyethylene, ethylene-vinyl acetate copolymer, polypropylene, polymethyl methacrylate, methacrylic-styrene copolymer, cellulose acetate, polyethylene terephthalate, vinylidene fluoride, and the like.

Examples of the photocurable resin include urethane acrylate, epoxy acrylate, polyester acrylate, polybutadiene acrylate, silicone acrylate, amino resin acrylate, alicyclic epoxy resins, glycidyl ether epoxy resins, urethane vinyl ether, polyester vinyl ether, and the like.

Among thermosetting resins and thermoplastic resins, there is a resin having a property of curing at room temperature. Examples of such room temperature setting resin include silicone resins, epoxy resins, phenol resins, polymethyl methacrylate, and the like, and these resins are particularly suitable as a covering agent used for a processed bone.

The adhesive as the covering agent can be appropriately selected from inorganic adhesives, natural adhesives, and synthetic adhesives. Examples of the inorganic adhesives include sodium silicate, cement, plaster, and the like.

Examples of the natural adhesives include natural rubber adhesives, casein adhesives, water resistant starch adhesives, glue, albumin, and the like.

Examples of the synthetic adhesive include epoxy resin-based adhesives, acrylic resin-based adhesives, α-olefin resin-based adhesives, polyethylene resin-based adhesives, polyvinyl acetate resin-based adhesives, vinyl chloride resin-based adhesives, ethylene-vinyl acetate resin-based adhesives, cyanoacrylate-based adhesives, aqueous polymer isocyanate-based adhesives, chloroprene rubber-based adhesives, styrene-butadiene rubber-based adhesives, nitrile rubber-based adhesives, polysulfide-based adhesives, butyl rubber-based adhesives, silicone rubber-based adhesives, polystyrene-based adhesives, polyvinyl acetate-based adhesives, modified silicone-based adhesives, polyolefin-based adhesives, polyurethane-based adhesives, polymethacrylate resin-based adhesives, phenol resin-based adhesives, urea resin-based adhesives, melamine resin-based adhesives, resorcinol-based adhesives, polyester-based adhesives, polyimide-based adhesives, nitrocellulose adhesives, methylcellulose, carboxymethyl cellulose, and the like. These synthetic adhesives may be in the form of a liquid or emulsion. Moreover, a tape obtained by applying an adhesive to an appropriate substrate, such as an acrylic resin-based pressure-sensitive adhesive tape, may be used.

The polymer membrane as the covering agent can be appropriately selected from a biopolymer membrane and a synthetic polymer membrane. Examples of the biopolymer membrane include polysaccharide membranes such as chitosan, alginate, and pectin membranes; plant-derived cellulose membranes such as regenerated cellulose and cellulose triacetate membranes, and the like. Further, a membrane obtained by alternately laminating chitosan and an alginate is suitable as the covering agent. Examples of the synthetic polymer membrane include polyacrylonitrile, polymethyl methacrylate, polysulfone, polyether sulfone, polyvinylidene chloride, polyvinyl chloride, medium-density polyethylene, low-density polyethylene, polypropylene, ethylene-vinyl alcohol copolymer membranes, and the like. The shape of the polymer membrane is not particularly limited, and it can be appropriately selected from a tape shape, a film shape, and a sheet shape according to the shape of the bone.

The gel as the covering agent may be appropriately selected from gels containing water as a solvent, and examples include agar, gelatin, agarose gel, polyacrylamide gel, polyhydroxyethyl methacrylate gel, and the like.

Gypsum as the covering agent contains calcium sulfate as a main ingredient. In the method of the present embodiment, hemihydrate gypsum, dihydrate gypsum, anhydrous gypsum or the like can be used. For example, a plaster cast which includes burnt gypsum powder and cotton cloth may be used as the covering agent.

The method of adhering the covering agent to the outer surface of the bone can be appropriately selected according to the type or form of the covering agent. For example, when using a covering agent that hardens from a liquid state to a solid state, the bone is immersed in the covering agent in the liquid state, or the covering agent in the liquid state is applied to the outer surface of the bone, etc. Then, coating the entire bone with a covering agent, and curing the covering agent in that state can be mentioned. When using a covering agent that cures from a plastic state such as putty, covering the entire bone with the covering agent in a plastic state and curing the covering agent in that state can be mentioned. When using a covering agent in the form of a thin film, covering the entire bone may be mentioned by applying the covering agent to the outer surface of the bone or covering the bone with the covering agent.

In the perfusion method of the present embodiment, first, a fluid containing an undifferentiated cell is introduced into the above-described organ or tissue. Thus, the undifferentiated cell is introduced into an organ or tissue. Here, the term “undifferentiated cell” refers to a cell other than terminally differentiated cell. The term “terminally differentiated cell” refers to a cell that has reached terminal differentiation in cell lineage. Therefore, the terminally differentiated cell does not differentiate any more. In the present specification, the terminally differentiated cell also includes a platelet.

The undifferentiated cell includes, for example, a stem cell and a precursor cell. The stem cell include an ES cell (Embryonic Stem cell), a cloned ES cell, an iPS cell (induced Pluripotent Stem cell), a MUSE cell (Multiliniage-differentiating Stress Enduring cell), a mesenchymal stem cell, a neural stem cell, an epithelial stem cell, a hepatic stem cell, a germ stem cell, a hematopoietic stem cell, a skeletal stem cell, and the like. The precursor cell includes a platelet precursor cell, a liver precursor cell, a heart precursor cell, a neuronal precursor cell, and the like. The platelet precursor cell includes a megakaryocyte precursor cell, a megakaryoblast, a promegakaryocyte, a megakaryocyte (mature megakaryocyte), and the like (hereinafter, these are also collectively referred to as “megakaryocytic cell”). The liver precursor cell includes a hepatoblast cell, a hepatic precursor cell, a hepatic stellate cell precursor cell, a hepatic stem precursor cell, a vascular endothelial precursor cell of liver, a mesothelial cell precursor cell of liver, and the like. The heart precursor cell includes a myocardial precursor cell, a vascular endothelial precursor cell of the heart, and the like. The neuronal precursor cell includes a neuron precursor cell, a glial precursor cell, a vascular endothelial precursor cell of the cerebral nervous system, and the like.

In a preferred embodiment, a megakaryocytic cell is used as the undifferentiated cell. The megakaryocytic cell can be obtained, for example, by stimulating a hematopoietic stem cell with cytokine or the like. When a fluid containing a megakaryocytic cell is used in the perfusion method of the present embodiment, a platelet can be obtained by differentiating the megakaryocytic cell in an organ or tissue. The platelet obtained by the perfusion method of the present embodiment has the same function as a platelet in a living body.

The fluid containing the undifferentiated cell can be prepared by containing the undifferentiated cell in a liquid capable of surviving or culturing the undifferentiated cell. Examples of such liquid include liquid media, organ preservation solutions, Ringer's solution, Krebs-Ringer solution, physiological saline, mixtures thereof, and the like. Hereinafter, these liquids are also collectively referred to as “perfusate”. Examples of the liquid medium include RPMI medium (Roswell Park Memorial Institute medium), MEM medium (Minimum Essential Media), DMEM medium (Dulbecco's Modified Eagle Medium), Ham's F-12 medium, and the like. The organ preservation solution includes Celsior solution, LPD (Low potassium dextran) solution, ET-Kyoto solution, Euro-Collins solution, UW (UNIVERSITY of Wisconsin) solution, and the like. The perfusate may optionally contain additives suitable for cell maintenance and the like, such as plasma, serum, amino acids, and the like, as needed.

The cell concentration in the fluid containing the undifferentiated cell is not particularly limited, and may be appropriately determined, for example, in the range of 1×10³ cells/mL or more and 1×10⁸ cells/mL or less. The amount of the fluid containing the undifferentiated cell is not particularly limited, and may be appropriately determined, for example, in the range of 0.1 mL or more and 50 mL or less. In particular, it is desirably the range of 0.5 mL or more and 3 mL or less.

In the present embodiment, the means for introducing a fluid containing an undifferentiated cell into an organ or tissue is not particularly limited. Examples of the means include pouring the fluid containing the undifferentiated cell from a site to which liquid is introduced inside the organ or tissue, by a tube connecting to a fluid feeding pump or an injection needle connecting to a syringe. Alternatively, a container containing the fluid containing the undifferentiated cell is connected to the site to which liquid is introduced in the organ or tissue, by way of a tube or the like, and the fluid may be sucked by a syringe or a fluid feeding pump from a site from which liquid in the organ or tissue is derived out. Alternatively, these methods may be combined. The fluid containing the undifferentiated cell may be introduced using the perfusion device of the present embodiment described above. When the fluid containing the undifferentiated cell is introduced into an organ, it is preferable to introduce it through blood vessels of the organ. When the fluid containing the undifferentiated cell is introduced into a processed bone, it is preferable to introduce it from a hole formed in the processed bone.

The flow velocity of the fluid containing the undifferentiated cell should be a flow velocity generally set in organ perfusion experiments. For example, it may be set appropriately from 0.01 mL/min or more and 100 mL/min or less, preferably 0.1 mL/min or more and 50 mL/min or less, and more preferably 1 mL/min or more and 20 mL/min or less. The temperature of the fluid containing the undifferentiated cell should be a temperature at which the undifferentiated cell can survive. Such temperature is, for example, 4° C. or more and 40° C. or less, preferably 20° C. or more and 38° C. or less, and particularly preferably 37° C.

In the present embodiment, it is preferable to introduce a fluid containing the undifferentiated cell into an organ or tissue under positive pressure. Here, the introduction of a fluid containing an undifferentiated cell under positive pressure includes introducing a fluid containing the undifferentiated cell into the organ or tissue in a state where positive pressure is applied to the organ or tissue. That is, the fluid containing the undifferentiated cell may be introduced into the organ or tissue while the pressure applied to the organ or tissue is increased according to the introduction of the fluid containing the undifferentiated cell. For example, the fluid containing the undifferentiated cell may be introduced by providing a time for applying a positive pressure inside the organ or tissue. Thus, the undifferentiated cell can be distributed throughout the organ or tissue, and the amount of the undifferentiated cell introduced into the organ or tissue can be increased. The pressure inside the organ or tissue is preferably a pressure at a level that does not damage the organ or tissue. When using a processed bone, a pressure gauge is installed in a conduit connected to the introduction hole, and a fluid containing an undifferentiated cell should be introduced such that the pressure is in the range of 5 kPa or more and 100 kPa or less, and preferably 10 kPa or more and 75 kPa or less.

The introduction under positive pressure can be performed by introducing a fluid containing an undifferentiated cell into the organ or tissue in a state where liquid is not derived out from the organ or tissue. For example, by closing the site from which liquid is derived out in the organ or tissue, the organ or tissue is in a state in which liquid is not derived out. More specifically, when the site to which liquid is introduced is an artery of an organ and the site from which liquid is derived out is a vein of the organ, the vein or a conduit connected thereto is closed. When a fluid containing an undifferentiated cell is introduced from the artery of the organ in this state, the pressure inside the organ increases. When using a processed bone, the lead-out hole or a conduit connected thereto is closed. When the fluid containing the undifferentiated cell is introduced from the introduction hole of the processed bone in this state, the pressure inside the processed bone increases. In the case where there is one hole formed in the processed bone, the pressure inside the processed bone is increased by introducing the fluid containing the undifferentiated cell into the holes. Thus, it is possible to introduce the fluid containing the undifferentiated cell by providing a time for applying a positive pressure inside the organ or tissue. The means for closing the site from which liquid is derived out is not particularly limited as long as the closed state can be released. For example, the site from which liquid is derived out may be closed with a stopper or a film, and a conduit connected to the site may be closed with a valve.

In the present embodiment, the direction of introducing the fluid containing the undifferentiated cell into an organ or tissue does not have to be unidirectional. For example, after the fluid containing the undifferentiated cell is once introduced from the introduction hole, a syringe or a fluid feeding pump may be operated to feed the fluid containing the undifferentiated cell in the reverse direction. The fluid containing the undifferentiated cell can be reciprocated in the organ or tissue by performing such operation once or repeating a plurality of times. Thus, the undifferentiated cell can be distributed throughout the organ or tissue, and the amount of undifferentiated cell introduced into the organ or tissue can be increased.

In the present embodiment, the organ or tissue may be perfused with perfusate prior to the introduction of the fluid containing the undifferentiated cell. Thus, the interior of the organ or tissue can be washed with the perfusate to remove contaminants such as cells present in the organ or tissue. Examples of means for introducing the perfusate include pouring the perfusate from the site into which liquid is introduced into the organ or tissue, by a tube connecting to a fluid feeding pump or an injection needle connecting to a syringe. Alternatively, a container containing the perfusate is connected to the site to which liquid is introduced in the organ or tissue, by way of a tube or the like, and the perfusate may be sucked by a syringe or a fluid feeding pump from a site from which liquid is derived out in the organ or tissue. Alternatively, these methods may be combined. The perfusate may be introduced using the perfusion device of the present embodiment described above.

The flow velocity of the perfusate is not particularly limited, and may be, for example, about the same as the flow velocity of the fluid containing the undifferentiated cell. The time of perfusion is not particularly limited, and is, for example, 1 minute or more and 50 hours or less, preferably 15 minutes or more and 25 hours or less, and more preferably 30 minutes or more and 10 hours or less. The temperature of the perfusate is not particularly limited, and is, for example, 4° C. or more and 50° C. or less, preferably 20° C. or more and 45° C. or less, and more preferably 22° C. or more and 42° C. or less. The particularly preferred temperature is 37° C.

In the present embodiment, in order to prevent drying of the organ or tissue during introduction and perfusion of the fluid containing the undifferentiated cell, the organ or tissue may be immersed in an appropriate liquid or placed still in the liquid. The liquid can be appropriately selected from the perfusate described above. When using a processed bone, it is not necessary to immerse in liquid.

In the perfusion method of the present embodiment, the undifferentiated cell is cultured in an organ or tissue into which a fluid containing the undifferentiated cell has been introduced. By culturing, the undifferentiated cell differentiates in the organ or tissue. Here, “differentiation of undifferentiated cell” and “undifferentiated cell differentiates” mean that differentiation of undifferentiated cell proceeds. Therefore, the differentiation of undifferentiated cell also includes not only undifferentiated cell (for example, megakaryoblast) becoming terminally differentiated cell (for example, platelet) but also undifferentiated cell (for example, megakaryoblast) becoming more differentiated undifferentiated cell (for example, megakaryocyte). That is, the cell differentiated from the undifferentiated cell may be a terminally differentiated cell or a cell that can be further differentiated.

The culture is preferably performed by maintaining a state that the introduced undifferentiated cell stays in the organ or tissue. For example, the organ or tissue into which the fluid containing the undifferentiated cell has been introduced may be allowed to stand under conditions suitable for culture of the undifferentiated cell. Moreover, when using the perfusion device of the present embodiment described above, introduction of the liquid to the organ or tissue may be stopped by stopping a fluid feeding pump or operating a switching valve. In the culture, these sites may be closed so that the fluid containing the undifferentiated cell does not leak from the sites to which liquid is introduced or derived out in the organ or tissue.

The conditions suitable for culture of the undifferentiated cell themselves are known in the art. The organ or tissue into which the fluid containing undifferentiated cell has been introduced may be placed in a CO₂ incubator or the like used for cell culture. The temperature conditions are, for example, 4° C. or more and 50° C. or less, preferably 20° C. or more and 45° C. or less, and more preferably 22° C. or more and 42° C. or less. The particularly preferred temperature is 37° C. The time of culture is not particularly limited, and is, for example, 10 minutes or more and 72 hours or less, preferably 1 hour or more and 48 hours or less, and more preferably 2 hours or more and 30 hours or less.

In the perfusion method of the present embodiment, after completion of the culture, a fluid containing a cell differentiated from the undifferentiated cells (hereinafter, also referred to as “a fluid containing a differentiated cell”) is recovered from the organ or tissue. The fluid containing the differentiated cell may contain a product of the cell differentiated from the undifferentiated cell. Such product may be a secretion of the differentiated cell or a degradation product of the differentiated cell.

A means for recovering the fluid containing the differentiated cell from the organ or tissue is not particularly limited. For example, by introducing a perfusate to a site into which liquid is introduced of the organ or tissue, the fluid containing the differentiated cell may be derived out from the site from which liquid is derived out of the organ or tissue. Alternatively, the fluid containing the differentiated cell may be sucked from the site from which liquid is derived out of the organ or tissue. For recovery of the fluid containing the differentiated cell, a tube connecting to a fluid feeding pump, an injection needle connecting to a syringe or the like may be used. Alternatively, the perfusion device of the present embodiment described above may be used. When the fluid containing the differentiated cell is recovered from an organ, it is preferable to recover it through blood vessels of the organ. When the fluid containing the differentiated cell is recovered from a processed bone, it is preferable to recover it from a lead-out hole formed in the processed bone.

When the fluid containing the differentiated cell is derived out and recovered by introduction of perfusate, the amount of the introduced perfusate is preferably an amount capable of sufficiently deriving the cells in the organ or tissue. Such amount should be, for example, 2 mL or more and 2000 mL or less as an amount capable of recovering the introduced fluid containing the undifferentiated cell. The flow velocity of the perfusate is not particularly limited, and may be, for example, about the same as the flow velocity of the fluid containing the undifferentiated cell. The temperature of the perfusate should be a temperature at which the differentiated cell can survive. Such temperature is, for example, 4° C. or more and 50° C. or less, preferably 20° C. or more and 45° C. or less, and more preferably 22° C. or more and 42° C. or less. The particularly preferred temperature is 37° C.

A further embodiment of the present invention provides a method of producing a platelet using the processed bone. In this method, first, the fluid containing the undifferentiated cell is introduced into the interior of a processed bone having an outer surface of the bone covered with a covering agent attached closely to the outer surface of the bone and a hole that penetrates through the covering agent and the outer surface of the bone and reaches the interior of the bone. In this embodiment, no pressure may be applied to the interior of the processed bone when introducing the undifferentiated cell. As the undifferentiated cell, an undifferentiated cell capable of differentiating into a platelet is preferable, and examples thereof include a megakaryocytic cell. The details of the processed bone used in this method and the means and conditions of introduction of the fluid containing the undifferentiated cell are as described above. Subsequently, the undifferentiated cell is cultured in the interior of the processed bone. The culture is preferably performed in a state in which the undifferentiated cell can be brought into contact with bone marrow. Details of culture conditions are as described above. Then, a fluid containing a platelet differentiated from the undifferentiated cell is recovered from the interior of the processed bone. Details of the means and conditions of recovery and the like are as described above.

Moreover, a further embodiment of the present invention provides a perfusion device including: a storage unit for disposing a processed bone having an outer surface of the bone covered with a covering agent attached closely to the outer surface of the bone, and a hole that penetrates through the covering agent and the outer surface of the bone and reaches the interior of the bone, a fluid feeding unit for feeding a fluid containing an undifferentiated cell, a recovery unit for recovering a fluid containing a platelet differentiated from the undifferentiated cell, a first conduit for connecting the processed bone disposed in the perfusion unit and the fluid feeding unit, and a second conduit for connecting the processed bone disposed in the perfusion unit and the recovery unit. In this perfusion device, the fluid feeding unit introduces a fluid containing a perfusate and/or undifferentiated cell into the processed bone through the first conduit. The recovery unit recovers the fluid containing the platelet derived out from the processed bone through the second conduit. Preferably, the processed bone has at least two holes. In this case, at least one hole may be an introduction hole for introducing liquid into the interior of the processed bone to be connected to the first conduit. Further, the remaining hole may be a lead-out hole for deriving out a liquid from the interior of the processed bone to be connected to the second conduit.

Hereinbelow, the present invention will be described in detail by examples, but the present invention is not limited to these examples.

### EXAMPLES

### Experimental Example 1: Differentiation Induction of Undifferentiated Cell by Perfusion Device Using Processed Bone

### (1) Materials

### (1.1) Preparation of Perfusion Unit

The femur of right hind leg was extracted from a pig (body weight: 14.6 kg, 2 months old) anesthetized with Ketalar. The obtained femur was covered with underwater epoxy putty of an epoxy resin-based adhesive (Cemedine Co., Ltd.), and allowed to stand for about 20 minutes to cure the epoxy putty. Two holes of 1.1 mm in diameter were formed in the epoxy putty-coated femur by an electric drill. The holes were formed near both epiphyses of the femur. Hereinafter, the formed holes are referred to as an introduction hole and a lead-out hole. An injection needle (18G, Terumo Corporation) was inserted into each of the introduction hole and the lead-out hole to obtain a processed bone to be used in a perfusion device.

### (1.2) Preparation of Perfusate

To RPMI-1640 medium Hepes modification (R5886, Sigma) was added 10% (final concentration) FBS (Hyclone), 50-fold diluted Antibiotic-Antimycotic (15240-062, Gibco), 2 mM (final concentration) L-Glutamin (G7513, Sigma) to prepare a perfusate.

### (1.3) Preparation of Megakaryocytic Cells

### (i) Differentiation Induction of CD34-Positive Hematopoietic Stem Cells

Human umbilical cord blood-derived CD34-positive hematopoietic stem cells (Human CB CD34⁺ Mixed, ST-70008, Veritas) were washed with PBS. The cells were inoculated into a human megakaryocyte differentiation induction medium (SFEMII, ST-09655, Veritas) containing a cytokine cocktail (StemSpan Megakaryocyte Expansion Supplement, ST-2696, Veritas) at a final concentration of 1%. Passages were performed every 3 or 4 days so that the cell density was 1×10⁵ to 1×10⁶ cells/mL. For passage, the medium containing the cells was centrifuged (300 g, 5 minutes, room temperature), the supernatant was discarded, and the above medium was added to the cells. By culture for 19 days, the CD34-positive hematopoietic stem cells were differentiated into megakaryocytic cells.

(ii) Staining of Megakaryocytic Cells with Fluorescent Dye

The medium containing the megakaryocytic cells obtained above was centrifuged (200 g, 10 minutes, room temperature), the supernatant was discarded, and 30 mL of PBS was added to the cells. After the same operation was further performed, 3 μL of a CFSE solution (1 mg/mL) was added (final concentration 0.1 μg/mL), and allowed to stand at 37° C. for 30 minutes. Thereafter, the fluid containing megakaryocytic cells was centrifuged (200 g, 10 minutes, room temperature), the supernatant was discarded, and 30 mL of PBS was added thereto. The fluid containing megakaryocytic cells was centrifuged (200 g, 10 minutes, room temperature), the supernatant was discarded, and the perfusate was added. Thus, CFSE-stained megakaryocytic cells were obtained.

### (2) Differentiation Induction of Megakaryocytic Cells

### (2.1) Perfusion

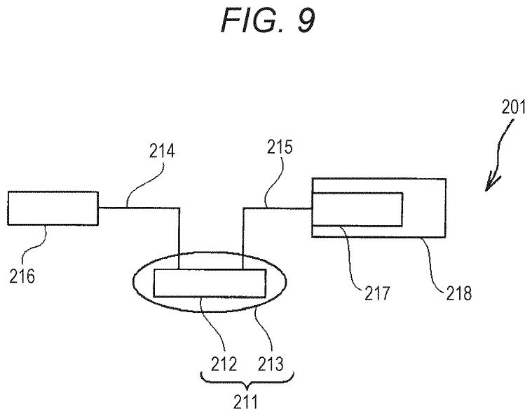

In Experimental Example 1, a perfusion device using the processed bone shown in FIG. 9 was formed. Here, the perfusion device will be described with reference to FIG. 9. A perfusion device 201 includes a processed bone 211 composed of a femur 212 and a covering agent 213, a first conduit 214 for introducing a perfusate into the processed bone, a second conduit 215 for deriving the perfusate from the processed bone, a syringe 216 storing the perfusate and a syringe 217 for recovering a fluid derived out from the processed bone. In FIG. 9, the syringe 217 is set to a syringe pump 218.

Specifically, the perfusion device 201 was formed as follows. A 50 mL syringe (Terumo Corporation) was connected to the injection needle inserted into the introduction hole by way of a tube (inner diameter 0.8 mm, Masterflex). Moreover, a 50 mL syringe set to a syringe pump (YMC. CO., LTD.) was connected to the injection needle inserted in the lead-out hole by way of a tube. Thus, the perfusion device 201 using a processed bone was formed. Hereinafter, the syringe connecting to the introduction hole is also called “syringe A”, and the syringe connecting to the lead-out hole is also called “syringe B”. 50 mL of perfusate was stored in the syringe A. The syringe B was pulled by setting the flow rate of the syringe pump at 7 mL/min to apply negative pressure to the processed bone. At this time, the syringe A was pushed to promote introduction of the perfusate as needed. Thereby, the perfusate in the syringe A enters the processed bone from the introduction hole, and is derived out to the syringe B from the lead-out hole. That is, the perfusate perfused in the processed bone. The syringe A was supplied with perfusate, and a total of 280 mL of perfusate was perfused.

### (2.2) Introduction and Culture of Cells

The 50 mL syringe connected to the introduction hole was replaced with a 10 mL syringe (Terumo Corporation) storing 3 mL of perfusate containing megakaryocytic cells (1.1×10⁶) and connected to the introduction hole. The syringe pump was operated, and negative pressure was applied to the processed bone until no perfusate containing megakaryocytic cells in syringe A was left. Thereby, the megakaryocytic cells were introduced into the processed bone. The processed bone into which the megakaryocytic cells were introduced was allowed to stand in a CO₂ incubator set at 37° C. for 2 hours, and the megakaryocytic cells were cultured inside the pig femur for 3 hours. As a control, a perfusate containing megakaryocytic cells at the same cell concentration was placed in a tube and cultured in a CO₂ incubator set at 37° C. for 3 hours.

### (2.3) Cell Recovery

After culture, a 50 mL syringe storing 30 mL of perfusate was connected to the introduction hole, and negative pressure was applied to the processed bone with the syringe pump to perfuse the perfusate. At this time, the syringe A was pushed to promote introduction of the perfusate. Thereby, the perfusate (30 mL) derived out from the lead-out hole was recovered into the syringe B. Cells in the perfusate were fixed by adding PFA/PBS to the recovered perfusate so that the final concentration of PFA was 1%. Cells cultured in an in vitro environment (in a tube) as a control were similarly fixed with PFA/PBS (final concentration 1%).

### (3) FCM Analysis

Platelets differentiated from the megakaryocytic cells were detected based on platelet surface markers CD42b and CD61. Specific operations are as follows. The perfusate containing the fixed cells was centrifuged (200 g, 10 minutes, room temperature), the supernatant was discarded, and 30 mL of PBS was added to the cells. The same operation was further performed. The fluid containing cells was centrifuged (1500 g, 10 minutes, room temperature), and the supernatant was discarded. The same operation was performed twice more. Thereafter, APC-labeled anti-CD42b antibody or Alexa 647-labeled anti-CD61 antibody was added at a ratio of 20 μL of antibody to 100 μL of a fluid containing cells, and an antigen-antibody reaction was performed for 30 minutes. As a negative control, an isotype antibody of the above antibody labeled with the same dye was used. After the reaction, the fluid containing cells was centrifuged (1500 g, 10 minutes, room temperature), the supernatant was discarded, and 1% BSA/PBS was added to the cells. For FCM analysis of immunostained cells, FACS Verse (BD) was used. In FCM analysis, first, CFSE-positive cells were extracted, then platelet-sized cells were extracted, and finally, CD42b-positive or CD61-positive cells were extracted. Thereby, CFSE-positive and CD42b-positive or CD61-positive platelets were extracted.

### (4) Results

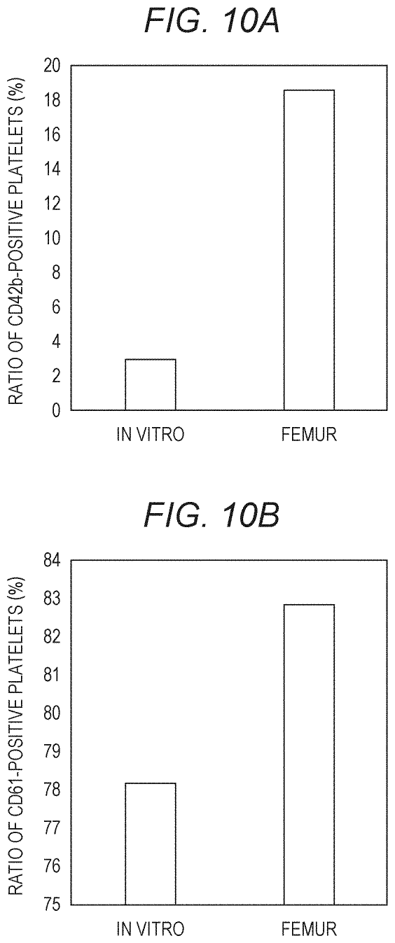

The results of FCM analysis are shown in FIGS. 10A and 10B. In the figures, “in vitro” indicates a cell cultured in a tube. As shown in FIGS. 10A and 10B, the ratio of CD42b-expressing cells in the platelet-sized cells derived from the megakaryocytic cells introduced into the processed bone was 18.7%, and the ratio of CD61-expressing cells was 82.9%. The concentration of CD42b-expressing cells in the recovered fluid was 201.3 cells/mL, and the concentration of CD61-expressing cells was 653.3 cells/mL. On the other hand, when megakaryocytic cells were cultured in an in vitro environment (in a tube), the ratio of CD42b-expressing cells in the platelet-sized cells was 3.0%, and the ratio of CD61-expressing cells was 78.2% (see FIGS. 10A and 10B). From these results, it was shown that it is possible to differentiate megakaryocytic cells into platelets by introducing megakaryocytic cells into processed bone and culturing the cells more efficiently than culturing in an in vitro environment (in a tube).

### Experimental Example 2: Differentiation Induction of Undifferentiated Cells by Perfusion Device Using Processed Bone (2)

In Experimental Example 2, differentiation induction and FCM analysis of megakaryocytic cells were performed in the same manner as in Experimental Example 1 except that 2 mL of perfusate containing megakaryocytic cells (8.0×10⁶) prepared separately from Experimental Example 1 was introduced into the processed bone.

As a result, the ratio of CD42b-expressing cells in the platelet-sized cells derived from the megakaryocytic cells introduced into the processed bone was 22.2%, and the ratio of CD61-expressing cells was 60.5%. On the other hand, when megakaryocytic cells were cultured in an in vitro environment (in a tube) as a control, the ratio of CD42b-expressing cells in the platelet-sized cells was 4.54%, and the ratio of CD61-expressing cells was 52.2%. From these results, it was shown that it is possible to differentiate megakaryocytic cells into platelets by introducing megakaryocytic cells into processed bone and culturing the cells more efficiently than culturing in an in vitro environment (in a tube).

### Experimental Example 3: Differentiation Induction of Undifferentiated Cells by Perfusion Device Using Spleen

### (1) Materials

### (1.1) Perfusion Unit

The spleen was extracted from a pig (body weight: 13.8 kg, 2 months old) anesthetized with Ketalar, and the short gastric artery was ligated. A TOP extension tube (X1-L75, TOP Corporation) was intubated in each of the splenic artery and splenic vein, and a three-way stopcock (type R 360°, Terumo Corporation) was attached to each tube, thereby obtaining a spleen to be used in a perfusion device. In Experimental Example 3, a tube intubated into the splenic artery was used as a pipe for introducing liquid into the spleen, and a tube intubated into the splenic vein was used as a pipe for deriving out liquid from the spleen. After intubation into the spleen, 500 mL of 1% (v/v) heparin-containing physiological saline was introduced from the splenic artery and derived out from the splenic vein to confirm that the spleen could be perfused. This also suppressed the blood clotting action in the spleen.

### (1.2) Perfusate and Megakaryocytic Cells

Perfusate and megakaryocytic cells were prepared in the same manner as in Experimental Example 1.

### (2) Differentiation Induction of Megakaryocytic Cells

### (2.1) Perfusion

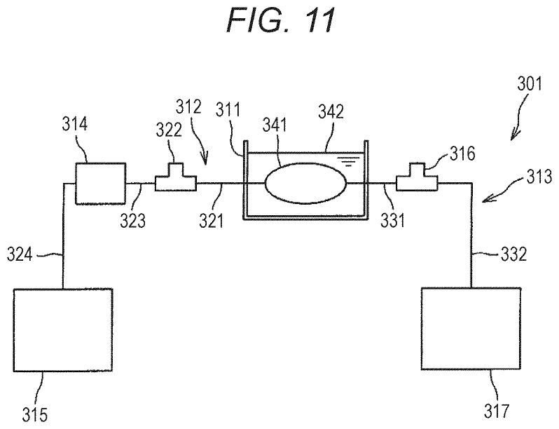

In Experimental Example 3, a perfusion device using the spleen shown in FIG. 11 was formed. Here, the perfusion device will be described with reference to FIG. 11. A perfusion device 301 includes a container 311 storing a spleen 341, a first conduit 312 for introducing perfusate into the spleen, a second conduit 313 for deriving out perfusate from an organ, and a fluid feeding pump 314 (Masterflex fluid feeding pump, 07528-10, Yamato Scientific Co., Ltd.), a container 315 storing perfusate, a three-way stopcock 316 as a switching valve for adjusting pressure inside the spleen, and a container 317 for recovering the derived fluid. In the container 311, the spleen 341 is allowed to stand in physiological saline 342. The first conduit 312 includes a tube 321 (the above TOP extension tube), a three-way stopcock 322, tubes 323 and 324 (inner diameter 4.8 mm, Masterflex). The first conduit 312 connects the spleen to the fluid feeding pump 314 and the container 315. The second conduit 313 includes a tube 331 (the above TOP extension tube described above), the three-way stopcock 316 and a tube 332 (inner diameter 4.8 mm, Masterflex). The second conduit 313 connects the spleen to the container 317 for recovering the derived fluid.

The perfusion device 301 was allowed to stand in a CO₂ incubator set at 37° C., and the fluid feeding pump 314 was operated. The perfusate was introduced from the splenic artery into the spleen and derived out from the splenic vein. Perfusion was performed for 3 hours by setting the flow velocity of the fluid feeding pump at about 10 mL/min.

### (2.2) Introduction and Culture of Cells

20 mL of perfusate containing megakaryocytic cells (5.0×10⁷) was stored in the container 315. The fluid feeding pump 314 was operated to introduce the perfusate containing megakaryocytic cells from the splenic artery into the spleen. At this time, the three-way stopcock 316 was operated to prevent the perfusate from being derived out of the tube 332. Since the perfusate is introduced into the spleen in a state in which the second conduit is closed by the three-way stopcock 316, the interior of the spleen has a positive pressure. After introducing the perfusate containing megakaryocytic cells, the three-way stopcock 322 was operated to prevent the perfusate from flowing back into the tube 321. This maintained positive pressure inside the spleen. Then, megakaryocytic cells were cultured under positive pressure by allowing the spleen into which the megakaryocytic cells were introduced to stand in the incubator for 3 hours. As a control, a perfusate containing megakaryocytic cells at the same cell concentration was placed in a tube and cultured in an incubator for 3 hours.

### (2.3) Cell Recovery

After culture, the perfusate was perfused to recover 120 mL of the perfusate derived out from the splenic vein. Then, cells in the perfusate recovered from the spleen and the tube were fixed, in the same manner as in Experimental Example 1.

### (3) FCM Analysis

Immunostaining and FCM analysis of the recovered cells were performed, in the same manner as in Experimental Example 1.

### (4) Results

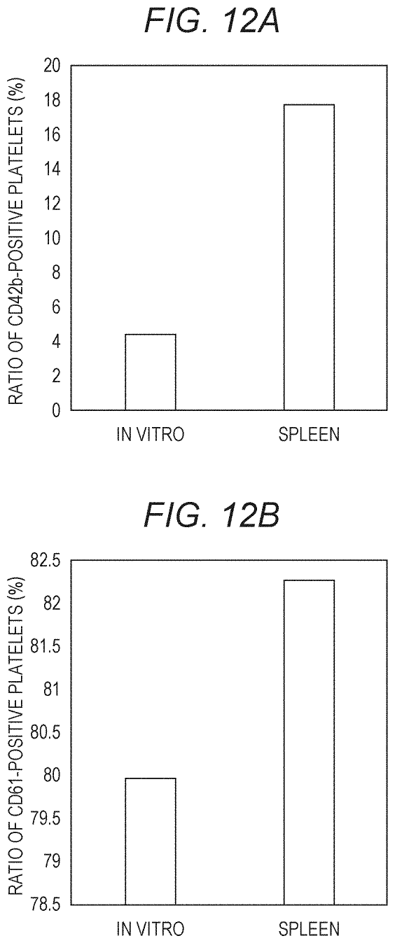

The results of FCM analysis are shown in FIGS. 12A and 12B. In the figures, “in vitro” indicates a cell cultured in a tube. As shown in FIGS. 12A and 12B, the ratio of CD42b-expressing cells in the platelet-sized cells derived from the megakaryocytic cells introduced into the spleen was 17.7%, and the ratio of CD61-expressing cells was 82.3%. On the other hand, when megakaryocytic cells were cultured in an in vitro environment (in a tube), the ratio of CD42b-expressing cells in the platelet-sized cells was 4.4%, and the ratio of CD61-expressing cells was 80.0% (see FIGS. 12A and 12B). From these results, it was shown that it is possible to differentiate megakaryocytic cells into platelets by introducing megakaryocytic cells into spleen and culturing the cells more efficiently than culturing in an in vitro environment (in a tube).

### Experimental Example 4: Differentiation Induction of Undifferentiated Cells by Perfusion Device Using Processed Bone (3)

### (1) Materials

In Experimental Example 4, a perfusion device using a processed bone prepared from a pig femur was prepared, in the same manner as in Experimental Example 1. In addition, perfusate and megakaryocytic cells were prepared in the same manner as in Experimental Example 1.

### (2) Differentiation Induction of Megakaryocytic Cells

### (2.1) Perfusion, Introduction and Culture of Cells

280 mL of perfusate was perfused into the processed bone, in the same manner as in Experimental Example 1. The 50 mL syringe connected to the introduction hole was replaced with a 10 mL syringe (Terumo Corporation) storing 2 mL of perfusate containing megakaryocytic cells (1.0×10⁷) and connected to the introduction hole. The tube connecting to the syringe B was closed by covering with parafilm to prevent the perfusate from being derived out from the lead-out hole. Then, the syringe A was pushed to introduce the perfusate containing megakaryocytic cells into the processed bone. Since the perfusate is introduced into the processed bone with the lead-out hole closed, the interior of the processed bone has a positive pressure. After the introduction of the perfusate containing megakaryocytic cells, the tube connecting the syringe A and the tube connecting to the syringe A was removed, and the tube was closed by covering with parafilm to prevent the perfusate from flowing back from the introduction hole or the introduced fluid from being dried. Then, megakaryocytic cells were cultured in the processed bone by allowing the processed bone into which the megakaryocytic cells were introduced to stand in the incubator for 3 hours. As a control, a perfusate containing megakaryocytic cells at the same cell concentration was placed in a tube and cultured in an incubator for 3 hours.

### (2.2) Cell Recovery

After culture, 60 mL of perfusate was perfused, and the perfusate (60 mL) derived out from the lead-out hole was recovered into the syringe B. Then, cells in the perfusate recovered from the spleen and the tube were fixed, in the same manner as in Experimental Example 1.

### (3) FCM Analysis

Immunostaining and FCM analysis of the recovered cells were performed, in the same manner as in Experimental Example 1. In addition, in order to analyze activation of platelets differentiated from the megakaryocytic cells, cells cultured in the processed bone in the same manner as in (2.1) and (2.2) above were recovered without being fixed. The recovered cells were stimulated with thrombin, stained with a labeled anti-PAC-1 antibody, and PAC-1-positive cells were detected by FCM analysis. Here, PAC-1 is a surface marker of activated platelets. In addition, cells not stimulated with thrombin were similarly analyzed. As a negative control, cells to which anti-PAC-1 antibody and RGDS which is a peptide that competes with the antibody were added were similarly analyzed.

### (4) Results

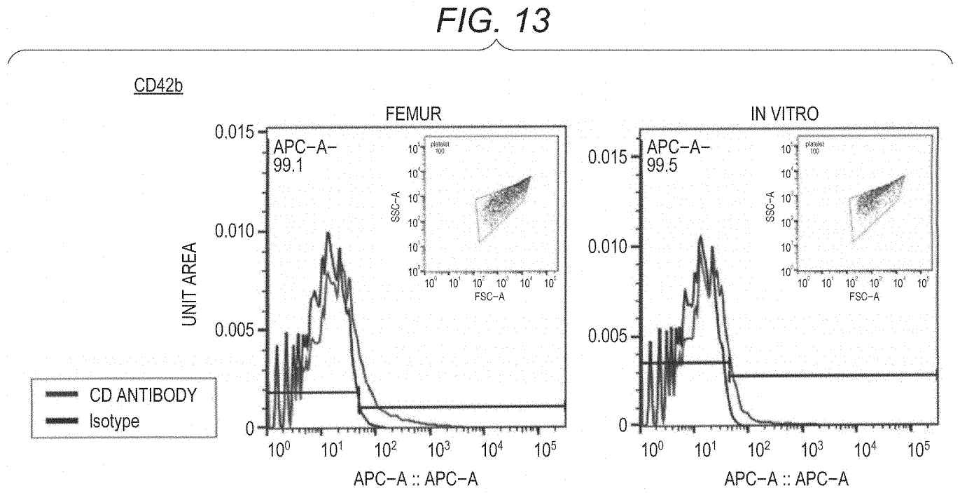

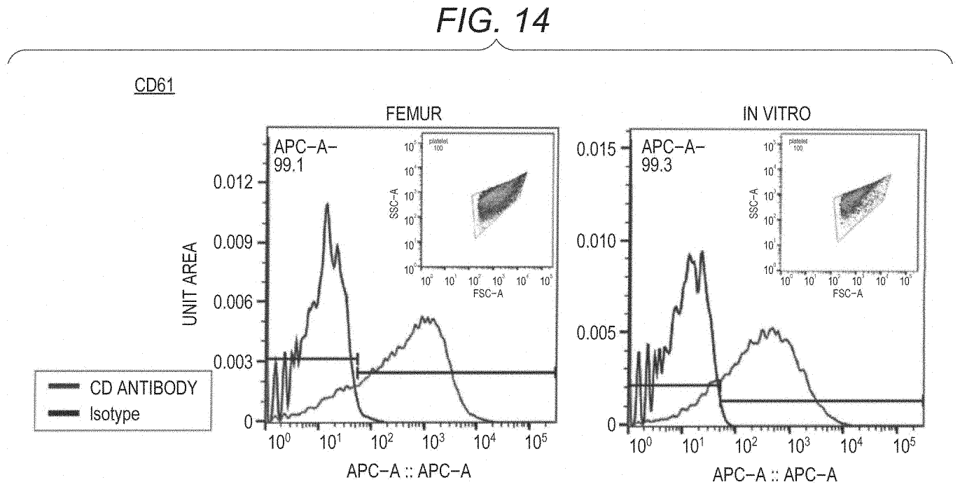

The results of FCM are shown in FIGS. 13, 14 and 15. In the graphs of these figures, the vertical axis indicates the number of cells, and the horizontal axis indicates fluorescence intensity. In FIG. 13, the black line is the result when the isotype antibody (negative control) was reacted, and the red line is the result when the anti-CD42b antibody was reacted. That is, from the result shown by the red line, the value obtained by subtracting the result shown by the black line (red line part having a fluorescence intensity of 5×10¹ or more) becomes a fluorescence signal derived from CD42b (cells expressing CD42b). A figure inserted in the upper right in FIG. 13 is a scattergram (horizontal axis: forward scattered light intensity, vertical axis: side scattered light intensity), and dots plotted in a gate (square on the scattergram) indicates CD42b-positive platelets. The ratio of CD42b-expressing cells in the platelet-sized cells derived from the megakaryocytic cells introduced into the processed bone was 10.0%. FIG. 14 shows the results obtained for CD61, and the vertical axis, the horizontal axis, and the inset (scattergram) in the graph of this figure are the same as FIG. 13. The ratio of CD61-expressing cells in the platelet-sized cells derived from the megakaryocytic cells introduced into processed bone was 76.3%.

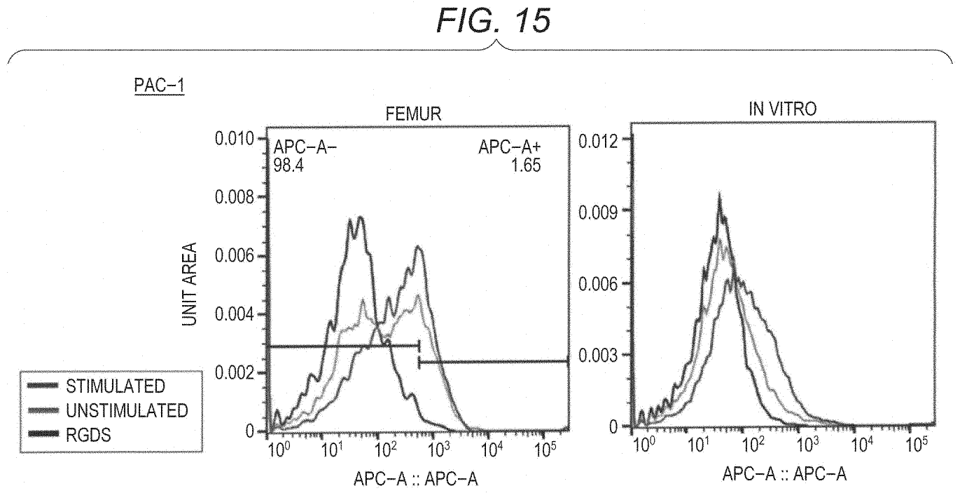

In FIG. 15, the black line is the result when RGDS (negative control) was reacted, and the light blue line is the result when the anti-PAC-1 antibody was reacted to a sample not subjected to thrombin stimulation, and the red line is the result when the anti-PAC-1 antibody was reacted after thrombin stimulation. That is, from the result shown by the red line, the value obtained by subtracting the result shown by the black line (red line part having a fluorescence intensity of 5×10² or more) becomes a fluorescence signal (cells expressing PAC-1) derived from platelets that expressed PAC-1 in response to thrombin stimulation. The ratio of PAC-1-positive cells when stimulated with thrombin was 25.0%. The concentration of CD42b-expressing cells in the recovered fluid was 1341 cells/mL, and the concentration of CD61-expressing cells was 9381 cells/mL. On the other hand, when megakaryocytic cells were cultured in an in vitro environment (in a tube), the ratio of CD42b-expressing cells in the platelet-sized cells was 5.8%, the ratio of CD61-expressing cells was 79.1%, and the ratio of PAC-1-expressing cells was 15.1%. From these results, it was shown that it is possible to differentiate megakaryocytic cells into platelets by introducing megakaryocytic cells into processed bone under positive pressure and then culturing the megakaryocytic cells in the processed bone more efficiently than culturing in an in vitro environment (in a tube). Moreover, compared with the result of Experimental Example 1, the number (concentration) of the produced platelets increased significantly by introducing megakaryocytic cells into processed bone under positive pressure.

### Experimental Example 5: Differentiation Induction of Undifferentiated Cells by Perfusion Device Using Processed Bone (4)

In Experimental Example 5, using processed bone prepared separately from Experimental Example 1, differentiation induction and FCM analysis of megakaryocytic cells were performed in the same manner as in Experimental Example 4 except that 2 mL of perfusate containing megakaryocytic cells (1.0×10⁷) or 3 mL of perfusate containing megakaryocytic cells (1.0×10⁷) was introduced into the processed bone.

When 2 mL of the perfusate was introduced, the ratio of CD42b-expressing cells in the platelet-sized cells derived from the megakaryocytic cells introduced into the processed bone was 12.7%, and the ratio of CD61-expressing cells was 76.5%. The concentration of CD42b-expressing cells in the recovered fluid was 3181 cells/mL, and the concentration of CD61-expressing cells was 18000 cells/mL.

When 3 mL of the perfusate was introduced, the ratio of CD42b-expressing cells in the platelet-sized cells derived from the megakaryocytic cells introduced into the processed bone was 13.0%, and the ratio of CD61-expressing cells was 78.1%. The concentration of CD42b-expressing cells in the recovered fluid was 1036 cells/mL, and the concentration of CD61-expressing cells was 6227 cells/mL.

As described above, in the same manner as the results of Experimental Example 4, the number (concentration) of the produced platelets increased significantly by introducing megakaryocytic cells into processed bone under positive pressure.

### Experimental Example 6: Measurement of Pressure in Processed Bone

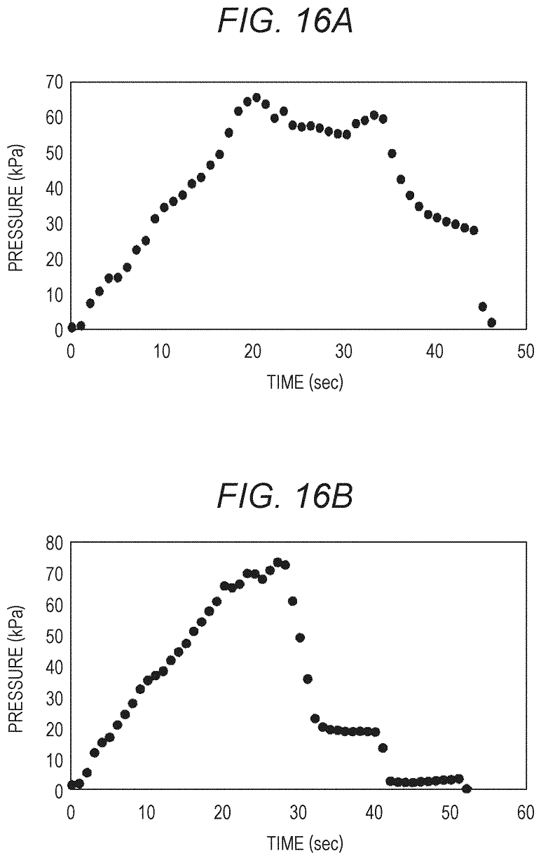

In Experimental Example 6, in the perfusion device of Experimental Example 1, a pressure gauge (Tem-Tech Lab.) was installed by way of a tube between the syringe A and the injection needle inserted into the introduction hole. Then, in a state in which the tube connecting to the syringe B was closed as in Experimental Example 4, 3 mL of perfusate was introduced into the processed bone. Fluctuations of pressure inside the processed bone between the start and end of the introduction of the perfusate were monitored. This experiment was performed twice. The results are shown in FIGS. 16A and 16B.

As shown in FIGS. 16A and 16B, the pressure increased when the introduction of the perfusate was started, and a pressure of 65 to 75 kPa was applied to the processed bone during the introduction. When no perfusate in the syringe A was left, the pressure dropped to 20 to 30 kPa. Then, when the syringe A was removed and the introduction hole was opened, the pressure instantaneously became 0 kPa. From these results, it could be confirmed that the processed bone has a positive pressure by introducing the perfusate in a state where the conduit on the lead-out hole side is closed.

### Experimental Example 7: Differentiation Induction of Undifferentiated Cells by Perfusion Device Using Processed Bone (5)

### (1) Materials

In Example 7, processed bone was prepared in the same manner as in Experimental Example 1 except that LOCTITE (trademark) quick-drying epoxy putty (Henkel Corporation) was used as a covering agent and that holes with a diameter of 1.3 mm were formed as an introduction hole and a lead-out hole. Further, perfusate and CFSE-stained megakaryocytic cells were prepared, in the same manner as in Experimental Example 1.

### (2) Differentiation Induction of Megakaryocytic Cells

### (2.1) Perfusion

In Experimental Example 7, perfusion was performed in the same manner as in Experimental Example 1 except that the syringe A was supplied with perfusate, and a total of 300 mL of perfusate was perfused into the processed bone.

### (2.2) Introduction and Culture of Cells

After washing CFSE-stained megakaryocytic cells with PBS, the cells were resuspended in 10 mL of perfusate to prepare a cell suspension of 1.48×10⁷ cells/10 mL. The 50 mL syringe connected to the introduction hole was replaced with a 10 mL syringe A (Terumo Corporation) storing 10 mL of perfusate containing megakaryocytic cells (1.48×10⁷) and connected to the introduction hole. Negative pressure was applied to the processed bone by operating and sucking a syringe pump attached to an empty syringe B connected to the lead-out hole at 7 mL/min. Thus, the fluid containing megakaryocytic cells in the syringe A was introduced into the processed bone. In the syringe B, a fluid containing megakaryocytic cells sucked from the lead-out hole was recovered. After no fluid containing megakaryocytic cells in the syringe A was left, the syringe pump was reconnected to the syringe A from the syringe B. The empty syringe A was sucked by the syringe pump, and a fluid containing megakaryocytic cells recovered into the syringe B was introduced into the processed bone from the lead-out hole. Thus, the operation of reconnecting the syringe pump and introducing the cell suspension under negative pressure was repeated five times. Thereby, the fluid containing megakaryocytic cells was reciprocated 2.5 times in the interior of the processed bone.

The processed bone into which the megakaryocytic cells were introduced was allowed to stand in a CO₂ incubator set at 37° C., and the megakaryocytic cells were cultured inside the pig femur for 3 hours. As a control, a fluid containing megakaryocytic cells at the same cell concentration was placed in a polypropylene tube and cultured in a CO₂ incubator set at 37° C. for 3 hours.

### (3) Analysis of Femoral Tissue

### (3.1) Preparation of Femoral Tissue Section

In Experimental Example 7, analysis of the femoral tissue was performed without recovering cells after culture. The femur after incubation was cut with an electric saw and bone marrow was scraped with a spatula. The bone marrow was embedded using OCT compound (Sakura Finetek Japan Co., Ltd.), and frozen blocks were prepared on dry ice. Frozen blocks of the tibia were also prepared as a control without megakaryocytic cells introduced. 10 μm thick bone marrow sections were prepared using cryosectioning (Leica), and immersed in physiological saline for 10 minutes to wash the OCT compound. The bone marrow sections were added with 4% PFA/PBS at 100 μL/section and fixed at room temperature for 10 minutes. The fixed bone marrow sections were immersed in physiological saline and washed, and then 1 μL of CD61-AF647 antibody (clone: VI-PL2, BioLegend, Inc.), and a mixed solution of 0.1 μL of Hoechst 33342 and 99 μL of PBS were added at 100 μL/section, and the resulting sections were stained at room temperature for 10 minutes. The stained bone marrow sections were immersed in physiological saline and washed, and then sealed with a 50% glycerol/PBS solution.

### (3.2) Counting of Megakaryocytes and Platelets

Tissue sections were observed using a fluorescence microscope (BZ-X700, KEYENCE CORPORATION). Megakaryocytes are CFSE positive (green) and CD61 positive (red). Therefore, a cell with a size of 10 μm to 70 μm that exhibits yellow to orange on the image and shows Hoechst 33342 positive (blue) was defined as a megakaryocyte. On the other hand, platelets were defined as tangible components with a size of 2 to 7 μm, exhibiting yellow to orange on the image and not showing Hoechst 33342 positive (blue).

### (4) Results

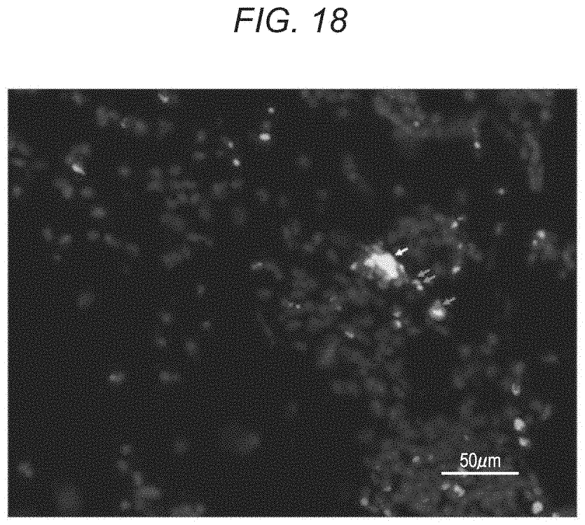

An example of the obtained image is shown in FIG. 18. In the figure, many platelets (gray arrows in the figure) were observed in the vicinity of megakaryocytes (white arrows in the figure). A plurality of regions were observed, and the number of megakaryocytes and platelets was counted, and the average number of platelets per megakaryocyte was found to be 4.27. Neither megakaryocytes nor platelets were observed in the frozen blocks of the tibia prepared as a control. On the other hand, when FCM analysis described in Experimental Example 1 was performed on a fluid containing megakaryocytic cells cultured in an in vitro environment (in a tube) as a control, only 0.012 platelets were produced per megakaryocyte. From the above, it was shown that it is possible to differentiate megakaryocytic cells into platelets by introducing megakaryocytic cells into processed bone and culturing the cells more efficiently than culturing in an in vitro environment (in a tube).

### Experimental Example 8: Differentiation Induction of Undifferentiated Cells by Perfusion Device Using Processed Bone (6)

### (1) Materials

Processed bone was prepared in the same manner as in Experimental Example 7. Further, perfusate and CFSE-stained megakaryocytic cells were prepared, in the same manner as in Experimental Example 1.

### (2) Differentiation Induction of Megakaryocytic Cells

### (2.1) Perfusion

The syringe A was supplied with perfusate, and a total of 300 mL of perfusate was perfused into the processed bone, in the same manner as in Experimental Example 7.

### (2.2) Introduction and Culture of Cells

The introduction and culture of cells were performed as follows in the same manner as in Experimental Example 4 except for the amount of megakaryocytic cells introduced. After washing CFSE-stained megakaryocytic cells with PBS, the cells were resuspended in 500 μL of perfusate to prepare a cell suspension of 3.6×10⁶ cells/500 μL. The 50 mL syringe connected to the introduction hole was replaced with a 1 mL syringe A (Terumo Corporation) storing 500 μL of perfusate containing megakaryocytic cells (3.6×10⁶) and connected to the introduction hole. The tube connecting to the syringe B was closed by covering with parafilm to prevent the perfusate from being derived out from the lead-out hole, in the same manner as in Experimental Example 4. Then, the syringe A was pushed to introduce the perfusate containing megakaryocytic cells into the processed bone. Since the perfusate is introduced into the processed bone with the lead-out hole closed, the interior of the processed bone has a positive pressure. After the introduction of the perfusate containing megakaryocytic cells, the tube connecting the syringe A and the tube connecting to the syringe A was removed, and the tube was closed by covering with parafilm to prevent the perfusate from flowing back from the introduction hole or the introduced fluid from being dried. Then, megakaryocytic cells were cultured in the processed bone by allowing the processed bone into which the megakaryocytic cells were introduced to stand in the incubator for 3 hours.

### (3) Analysis of Femoral Tissue

The femoral tissue sections were prepared and analyzed in the same manner as in Experimental Example 7.

### (4) Results

When an image of the tissue section (not shown) was observed, many megakaryocytes and platelets were observed as in Experimental Example 7. As a result of counting, platelets averaged 5.07 per megakaryocyte. Neither megakaryocytes nor platelets were observed in the frozen blocks of the tibia prepared as a control. From the above, it was shown that it is possible to differentiate megakaryocytic cells into platelets by introducing megakaryocytic cells into processed bone and culturing the cells more efficiently than culturing in an in vitro environment (in a tube).

What is claimed is:

1. A perfusion device comprising:
a storage unit in which an organ or tissue extracted from a living body is stored;
a fluid feeding unit configured to feed a fluid containing an undifferentiated cell into the organ or tissue stored in the storage unit;
a recovery unit configured to recover a fluid containing a cell differentiated from the undifferentiated cell;
a first conduit connecting the organ or tissue stored in the storage unit and the fluid feeding unit;
a second conduit connecting the organ or tissue stored in the storage unit and the recovery unit; and
a pressure regulating unit provided in the second conduit,
wherein the pressure regulating unit regulates pressure inside the organ or tissue to be positive when the fluid feeding unit feeds the fluid containing the undifferentiated cell into the organ or tissue.

2. The perfusion device according to claim 1, wherein the pressure regulating unit comprises a valve that adjusts a flow velocity or flow rate in the second conduit.

3. The perfusion device according to claim 1, wherein the pressure regulating unit regulates the pressure inside the organ or tissue to a pressure of 5 kPa or more and 100 kPa or less when the fluid feeding unit feeds the fluid containing the undifferentiated cell into the organ or tissue.

4. The perfusion device according to claim 1, wherein the pressure regulating unit regulates the pressure to prevent liquid from being derived out from the organ or tissue when the fluid feeding unit feeds the fluid containing the undifferentiated cell into the organ or tissue.

5. The perfusion device according to claim 1, wherein a pressure gauge is provided in the first conduit.

6. The perfusion device according to claim 1, wherein the organ is a solid organ extracted from an animal excluding human.

7. The perfusion device according to claim 1, wherein the tissue is a processed bone having an outer surface of a bone covered with a covering agent that adheres to the outer surface of the bone, and a hole that penetrates through the covering agent and the outer surface of the bone and reaches an interior of the bone.

8. The perfusion device according to claim 1, wherein the undifferentiated cell is a megakaryocytic cell, and the cell differentiated from the undifferentiated cell is a platelet.

9. The perfusion device according to claim 1, wherein the fluid feeding unit is configured to be able to perform reverse fluid feed.

10. A perfusion device comprising:
a storage unit in which an organ or tissue extracted from a living body is stored;
a first fluid feeding unit configured to feed a fluid containing an undifferentiated cell into an organ or tissue stored in the storage unit;
a recovery unit configured to recover a fluid containing a cell differentiated from the undifferentiated cell;
a first conduit connecting the organ or tissue stored in the storage unit and the first fluid feeding unit;
a second conduit connecting the organ or tissue stored in the storage unit and the recovery unit; and
a second fluid feeding unit provided in the second conduit,
wherein the second fluid feeding unit feeds the fluid containing the undifferentiated cell derived out from the organ or tissue into the organ or tissue by reverse fluid feed.

11. A perfusion method comprising:
feeding, under positive pressure, a fluid containing an undifferentiated cell into an organ or tissue extracted from a living body;
culturing the undifferentiated cell inside the organ or tissue; and
recovering a fluid containing a cell differentiated from the undifferentiated cell from the organ or tissue.

12. The perfusion method according to claim 11, wherein the fluid containing the undifferentiated cell is fed into the organ or tissue under a pressure of 5 kPa or more and 100 kPa or less.

13. The perfusion method according to claim 11, wherein the fluid containing the undifferentiated cell is fed into the organ or tissue in a state where liquid is not derived out from the organ or tissue.

14. The perfusion method according to claim 11, wherein the undifferentiated cell is a megakaryocytic cell, and the cell differentiated from the undifferentiated cell is a platelet.

15. The perfusion method according to claim 11, wherein the organ is a solid organ extracted from an animal excluding human.

16. The perfusion method according to claim 15, wherein the organ is selected from spleen, heart, liver, lung, kidney and pancreas.

17. The perfusion method according to claim 11, wherein the tissue is a bone extracted from an animal excluding human.

18. The perfusion method according to claim 17, wherein the tissue is a processed bone having an outer surface of a bone covered with a covering agent that adheres to the outer surface of the bone, and a hole that penetrates through the covering agent and the outer surface of the bone and reaches an interior of the bone.

19. The perfusion method according to claim 17, wherein the bone is selected from femur, humerus, sternum, pubis, ilium, rib, and vertebra.

20. The perfusion method according to claim 17, wherein the bone is femur extracted from a pig, and an amount of the fluid containing the undifferentiated cell to be fed into the femur is in a range of 0.5 mL or more and 3 mL or less.

21. The perfusion method according to claim 18, wherein the covering agent is at least one selected from a resin, an adhesive, a polymer membrane, a gel and gypsum.

22. A perfusion method comprising:
feeding a fluid containing an undifferentiated cell into an interior of a processed bone having an outer surface of a bone covered with a covering agent that adheres to the outer surface of the bone, and a hole that penetrates through the covering agent and the outer surface of the bone and reaches an interior of the bone;
culturing the undifferentiated cell in the interior of the processed bone; and
recovering a fluid containing a platelet differentiated from the undifferentiated cell from the interior of the processed bone.

23. The perfusion method according to claim 22, wherein the fluid containing the undifferentiated cell is fed into the interior of the processed bone by reciprocating the fluid in the interior of the processed bone.
